(19) United States
(12) Patent Application Publication
(10) Pub. No.: US 2024/0058531 A1
(43) Pub. Date: Feb. 22, 2024

(54) Electrode System, Electronic System, Drug Delivery Device, and a Method of Manufacturing an Electronic System

(71) Applicant: Sanofi, Paris (FR)

(72) Inventors: Alexander Allerdings, Frankfurt am Main (DE); Jonathan Jason Drake, Warwick (GB); Adam Moyo Harvey-Cook, Billericay Essex (GB); Oliver Charles Gazeley, Basel (CH)

(21) Appl. No.: 18/267,025

(22) PCT Filed: Dec. 15, 2021
(86) PCT No.: PCT/EP2021/085890

§ 371 Date: Jun. 13, 2023

(30) Foreign Application Priority Data
Dec. 16, 2020 (EP) 20315492.7

(51) Int. Cl. A61M 5/172

(52) U.S. Cl. CPC A61M 5/172; A61M 2207/10; A61M 2205/3317

(57) ABSTRACT

The present disclosure relates to a sensor electrode system for an electronic system. The sensor electrode system includes: a flexible conductor carrier which is electrically insulating; and an electrode arrangement. The electrode arrangement includes at least two electrically conductive electrode tracks. The electrically conductive electrode tracks extend along the flexible conductor carrier. The electrically conductive electrode tracks are electrically separated from one another along the conductor carrier. One of the electrically conductive electrode tracks forms a sensing electrode, and the sensing electrode extends in a sensing region of the flexible conductor carrier. Furthermore, an electronic system, a drug delivery device and a method are disclosed.

### CROSS REFERENCE TO RELATED APPLICATIONS

The present application is the national stage entry of International Patent Application No. PCT/EP2021/085890, filed on Dec. 15, 2021, and claims priority to Application No. EP 20315492.7, filed on Dec. 16, 2020, the disclosures of which are incorporated herein by reference.

### TECHNICAL FIELD

The present disclosure relates to an electrode system, an electronic system, a drug delivery device, and a method of manufacturing an electronic system.

### BACKGROUND

Drug delivery devices utilizing electronics are becoming increasingly popular within the industry and also with the patients or users.

### SUMMARY

It is an object of the present disclosure to facilitate improvements associated with electronic systems for drug delivery devices.

This object is achieved by the subject matter of the independent claims. Advantageous embodiments and refinements are subject to dependent claims. The present disclosure is not restricted to the currently claimed subject-matter. Rather, this disclosure may also cover currently unclaimed subject-matter which, however, could provide improvements and/or be made subject to the claims as will be readily appreciated by the skilled reader.

One aspect of the disclosure relates to an electrode system for a drug delivery device or for an electronic system. Another aspect relates to an electronic system, preferably one comprising the electrode system. The electronic system may be an electronic system for a drug delivery device. Yet another aspect relates to a drug delivery device, where the device preferably comprises the electrode system or the electronic system. Finally, the present disclosure relates to a method of manufacturing or assembling an electronic system for a drug delivery device.

In one embodiment, the electrode system comprises a flexible conductor carrier. The flexible conductor carrier may be electrically insulating. The flexible conductor carrier may be configured to provide a base for one or more electrically conductive electrode tracks running along the flexible conductor carrier. The respective electrode track may be flexible as well, either only in regions or in its entirety. The flexible conductor carrier and/or the electrode system may be deformed, e.g. folded, bent or curved, when applied in the electronic system. Thus, in the electronic system, the electrode system and/or the conductor carrier may have a second or deformed configuration, e.g. as opposed to a first or non-deformed configuration. In the first configuration, the conductor carrier may be flat. Having a flexible conductor carrier makes the conductor carrier and/or the entire electrode system easily adjustable to the specific requirements of the electronic system, in which it is put to use. For example, the flexible conductor carrier may be easily conformed to a non-plane, e.g. curved, surface, such as a cylindrical surface. Different regions of the conductor carrier may be deflected in different directions such that these regions face in different directions in the electronic system. Having a flexible conductor carrier is particularly suitable in electrode systems for drug delivery devices, such as injection devices and/or pen-type devices, as such devices usually have only limited space available such that having a flexible conductor carrier is particularly advantageous.

In one embodiment, the electrode system comprises an electrode arrangement. The electrode arrangement may extend along an electrode surface of the flexible conductor carrier. The electrode surface may be a main surface of the flexible conductor carrier. The electrode arrangement may be restricted to the electrode surface. The surface opposite from the electrode surface or facing away from that surface may be an electrically insulating surface. The insulating surface may be provided by the flexible conductor carrier. In this way, the electrode functionality of the electronic system may be provided on one surface, the electrode surface, and the opposite surface may be electrically insulated from the electrode arrangement which facilitates applying the electrode system in the use situation, e.g. in the electronic system, since the risk of short-circuits using elements arranged on that side of the conductor carrier facing away from the electrode surface is reduced on account of the insulating nature of the flexible conductor carrier and the insulating surface it provides. Alternatively to being insulating in its entirety, the surface of the conductor carrier opposite from the electrode surface or facing away from that surface may be a surface with one or more conductive electrode portions being exposed (that is to say two opposite surfaces of the conductor carrier may be electrode surfaces).

In one embodiment, the electrode arrangement comprises at least one electrically conductive electrode track. The electrode arrangement may comprise at least two electrically conductive electrode tracks, e.g. two tracks, three tracks or four tracks. The respective track may be formed or arranged on the electrode surface of the flexible conductor carrier. The respective electrically conductive electrode track may extend along the flexible conductor carrier. In case there is a plurality of electrically conductive electrode tracks, the electrically conductive electrode tracks may be electrically separated or insulated from one another along the conductor carrier. In other words, there may be no electrical interconnection between two electrically conductive electrode tracks, preferably between any arbitrarily selected pair of two electrically conductive electrode tracks, of the electrode arrangement on the conductor carrier.

In one embodiment, the electrode system is a sensor electrode system. The sensor electrode system may be configured to provide operative connection between a sensing site, e.g. an exterior operation surface of the user interface member, and a sensor controller (see further below), where the sensor controller is configured to evaluate one or more electrical characteristics input to the sensor controller via the electrode system. For example, the sensor electrode system, if applicable in combination with the sensor controller, is configured to sense capacitance or a change in capacitance on or close to an exterior operation surface of a user interface member of the drug delivery device or the electronic system. Specifically, the sensor electrode arrangement may be configured to sense the proximity of a user's finger(s), e.g. the thumb, a finger different from the thumb and/or at least the thumb and one further finger such as the index finger, to the exterior operation surface.

In one embodiment, one of the electrically conductive electrode tracks may form, may define or may be a sensing electrode. The sensing electrode may extend in a sensing region of the flexible conductor carrier. The sensing region may be a region of the conductor carrier which is configured to be arranged close to the exterior operation surface, preferably as close as possible. The exterior operation surface may be a setting surface, that is to say a surface which is touched by the user during or for a dose setting operation, or a delivery surface, that is to say a surface which is arranged to be touched by the user during or for a dose delivery operation.

In one embodiment, the electrode tracks may be configured to be connected with or form separate channels or inputs of a sensor controller which monitors one or more electrical characteristics in the sensing region by way of the one or more electrode tracks, e.g. the capacitance between two electrode tracks. Alternatively or additionally, the electrode tracks may be assigned to different sensing regions of the conductor carrier.

In one embodiment, the electrode system, the flexible conductor carrier, and/or the electrode arrangement, is deformable, e.g. in its entirety or at least in one or more regions. Preferably, the electrode system, the flexible conductor carrier, and/or the electrode arrangement is elastically deformable, e.g. in its entirety or at least in one or more regions. Therefore, the electrode system may be deformed, e.g. from a non-deformed configuration, into a deformed configuration. The deformation may result in an elastic restoring force which tends to reestablish the non-deformed configuration. The elastic restoring force may be used advantageously. For example, the elastic restoring force may be used to maintain contact of a portion of the flexible conductor carrier which is elastically deformed with another part, such as an inner wall of the user interface member, preferably facing away from the exterior operation surface. The elastic restoring force may press the surface of the flexible conductor carrier, e.g. the electrode surface, against the other part. Thus, the elastic deformability facilitates to conform the electrode system to a non-plane or curved, e.g. cylindrical, surface. The electrode system may be elastically deformable in its entirety or only one or more regions may be elastically deformable. The remaining region(s) of the electrode system may still be deformable but not necessarily elastically. A deformable electrode system, e.g. a flexible printed circuit board, has only small space requirements and is easily adjustable to different shapes and sizes of user interface members.

In one embodiment, one of the electrically conductive electrode tracks of the electrode arrangement is or forms a reference electrode. The reference electrode may extend along the sensing electrode in the sensing region of the flexible conductor carrier. The electrode track for the reference electrode may extend along the entire electrode track for the sensing electrode. The reference electrode and the sensing electrode may be configured to be provided with different electrical potentials. By way of the different potentials an electrical field may be formed between the reference electrode and the sensing electrode. The field may be static or dynamic, e.g. with an alternating potential or voltage. The electrical field or characteristics thereof may be monitored by a sensor controller of the system. Variations in the field or the characteristics, e.g. the capacitance, may be caused by the user's finger being close to the sensing and/or reference electrode. Therefore, variations may be evaluated by the sensor controller and, if they meet one or more predetermined criteria, be regarded as being caused by the user's finger being close to the exterior operation surface. For example, one of the electrodes may be provided with a high electrical potential and the other one may be provided with a low potential, e.g. ground potential. One potential may be positive and the other one may be negative. The low potential electrode may be the reference electrode, the high potential electrode may be the sensing electrode. The electronic system, e.g. the sensor controller, may be configured to monitor changes in capacitance between the reference electrode and the sensing electrode. If the change crosses a threshold level, e.g. becomes lower or greater than a predetermined threshold, the sensor controller may be configured to generate or provide a signal, e.g. to a main controller or electronic control unit of the electronic system. In case a touch event or a proximity event relative to the exterior operation surface occurs, the sensor controller may cause an according signal to be issued which can be used in the electronic system to change the operational state of the electronic system. In response to the signal, which may be a use signal or proximity signal, the electronic system may be configured to be switched from a first state of lower power consumption, e.g. a state where only the sensor controller is operational, to a second the state of higher power consumption, e.g. where one or more units of electronic system become operational which could not be operated previously. These units may include a motion sensing unit and/or a communication unit. The switching operation may be performed by an electronic control unit, e.g. the main controller, which may be provided in addition to the sensor controller.

In one embodiment, particularly in the sensing region of the flexible conductor carrier, the sensing electrode has a plurality of sensing electrode portions. The sensing electrode portions may be distributed over the sensing region, e.g. evenly. The sensing electrode portions may be separated from each other in the sensing region. The sensing electrode portions may be oriented parallel to one another. Alternatively or additionally, the reference electrode, particularly in the sensing region, may have a plurality of reference electrode portions. The reference electrode portions may be distributed over the sensing region, e.g. evenly. The reference electrode portions may be separated from one another in the sensing region. The reference electrode portions may be oriented parallel to one another in the sensing region. The respective electrode portion may be formed by the associated electrically conductive electrode track. The respective electrode portion may be oriented axially, e.g. in the electronic system, such as from the proximal end of the electronic system to the distal end of the electronic system. The sensing electrode portions in the sensing region may be equidistantly disposed along the sensing region. That is to say, any pair of two succeeding sensing electrode portions may have the same distance. The same may hold for the reference electrode portions, alternatively or additionally. Alternatively, distances between adjacent sensing electrode portions may vary, e.g. in a regular pattern. The same may hold for adjacent reference electrode portions, alternatively or additionally.

In one embodiment, one of the reference electrode and the sensing electrode has electrode portions, e.g. the reference electrode has reference electrode portions, which are oriented in opposite directions, e.g. opposite axial directions, in the sensing region. The electrode portions may have ends which face in different directions, e.g. different axial directions. Some electrode portions may face proximally, others may face distally. The other one of the reference electrode and the sensing electrode, e.g. the sensing electrode, may be arranged between two portions of the one of the reference electrode and the sensing electrode which portions are oriented in opposite directions.

In one embodiment, the respective electrode portion, i.e. reference electrode portion and/or sensing electrode portion, is axially oriented. The axis may be perpendicular to a longitudinal direction of main extension of the flexible conductor carrier. The respective electrode portion may have a free end.

In one embodiment, the sensing electrode portions and the reference electrode portions are alternatingly disposed in and/or along the sensing region, e.g. as seen along a main extension direction of the sensing region, the electrode system and/or the flexible conductor carrier. That is to say, a sensing electronic portion may be followed by a reference electrode portion which may be followed by another sensing electrode portion, which again, may be followed by another reference electrode portion.

In one embodiment, by way of the electrode portions, the sensing electrode and the reference electrode may have or define an interleaved or comb-like arrangement of the sensing electrode and the reference electrode in the sensing region. Here, as seen along the main extension direction, successive electrode portions may belong to different electrodes, i.e. to reference electrode or sensing electrode. A first comb may be formed by the sensing electrode and a second comb, which may be engaged with the first comb, may be formed by the reference electrode. The electrode portions may form the teeth of the respective comb. The reference electrode portions and/or the sensing electrode portions may be sequentially disposed along the sensing region, e.g. as seen along the main extension direction of the sensing region such as the main extension direction of the flexible conductor carrier.

In one embodiment, the distance between successive sensing electrode portions may be greater than the width of the reference electrode portion arranged between the adjacent sensing electrode portions. The same may hold for the distance between successive reference electrode portions which is expediently greater than the width of the sensing electrode portion arranged between the two adjacent reference electrode portions. The width may mean the dimension perpendicular to the extension of the electrode portion, e.g. towards an end of the electrode portion.

In one embodiment, the sensing electrode portions and/or the reference electrode portions are oriented obliquely or perpendicularly relative to the main extension direction of the sensing region, the electrode system and/or the flexible conductor carrier.

In one embodiment, the sensing region may be configured such that it is arranged close to the exterior operation surface, e.g. at a distance, preferably maximum distance, less than one of the following values: 2 mm, 1.5 mm, 1 mm. The exterior operation surface may be a circumferentially or angularly disposed surface, e.g. the setting surface of the user interface member. The main extension direction of the sensing region may be the direction along the circumferentially disposed surface, e.g. the angular direction, when the electrode system is provided in the electronic system.

In one embodiment, the sensing electrode portions have the same widths and/or the same lengths. Alternatively or additionally, the reference electrode portions may have the same widths and/or the same lengths. The width of the reference electrode portions may be equal to the width of the sensing electrode portions. The length or axial extension of the reference electrode portions may be equal to the length or axial extension of the sensing electrode portions.

In one embodiment, the sensing electrode portions are connected to a connecting portion of the sensing electrode. Preferably, all sensing electrode portions are connected to a common connecting portion, e.g. directly. The connecting portion may conductively interconnect the sensing electrode portions. The connecting portion may be a portion of the electrode track from which the sensing electrode portions branch off. All sensing electrode portions may branch off the connecting portion in the same direction. Transition regions between the connecting portion and the sensing electrode portions may be sequentially disposed along the connecting portion, e.g. linearly and/or along the main extension direction of the connecting portion. The respective sensing electrode portion may have an end which is remote from the connecting portion of the sensing electrode. The sensing electrode portions may extend linearly away from the connecting portion. The connecting portion may extend linearly along the flexible conductor carrier.

In one embodiment, the reference electrode portions are connected to a connecting portion of the reference electrode. Preferably, all reference electrode portions are connected to a common connecting portion, e.g. directly. The connecting portion may conductively interconnect the reference electrode portions. The connecting portion may be a portion of the electrode track from which the reference portions branch off. All reference portions may branch off the connecting portion in the same direction. Transition regions between the connecting portion and the reference portions may be sequentially disposed along the connecting portion, e.g. linearly.

The respective reference electrode may have an end which is remote from the connecting portion of the reference electrode. The reference electrode portions may extend linearly away from the connecting portion. The connecting portion may extend linearly along the flexible conductor carrier.

In one embodiment, the end, e.g. the free end, of at least one of the reference electrode portions, e.g. a plurality of reference electrode portions or all reference electrode portions, faces towards the sensing electrode or the electrode track for the sensing electrode, particularly as see along the flexible conductor carrier.

In one embodiment, the end, e.g. the free end, of at least one of the sensing electrode portions, e.g. a plurality of sensing electrode portions or all sensing electrode portions, faces towards the reference electrode or the electrode track for the reference electrode, particularly as see along the flexible conductor carrier.

In one embodiment, the number of the sensing electrode portions and/or the number of the reference electrode portions is even or odd. The number of sensing electrode portions may equal the number of reference electrode portions.

In one embodiment, the number of sensing electrode portions and/or the number of reference electrode portions is greater than or equal to one of the following values: 2, 3, 4, 5, 6, 7, 8.

In one embodiment, ends of the reference electrode portions, particularly all ends of the reference electrode portions, face towards the connecting portion of the sensing electrode. Alternatively or additionally, ends of the sensing electrode portions, particularly all ends of the sensing electrode portions, face towards the connecting portion of the reference electrode.

In one embodiment, the respective sensing electrode portion and/or the respective reference electrode portion extends linearly. Reference electrode portions and sensing electrode portions may be oriented such that they extend parallel to one another.

In one embodiment, one or a plurality of the respective electrode portions, i.e. reference electrode portions and/or sensing electrode portions, has a constant width along its extension, e.g. as seen in the direction away from the connecting portion connecting the respective electrode portions towards the free end of the respective electrode portion. The respective electrode portion may have a rectangular shape.

In one embodiment, one or a plurality of the respective electrode portions, i.e. reference electrode portions and/or sensing electrode portions, has a varying width along its extension, e.g. as seen in the direction away from the connecting portion connecting the respective electrode portions towards the free end of the respective electrode portion. The width of the electrode portion may decrease, preferably continuously, in the direction towards the free end. The free end may be a pointed end or a flat end. The respective electrode portion may have a triangle-like shape or a trapezoidal shape.

In one embodiment, the sensing region of the flexible conductor carrier is configured to conform to and/or extend along a circumferentially or angularly disposed sensing surface of a user interface member. When the electrode system is applied or included in the electronic system, the sensing region conforms to and/or extend along the sensing surface of the user interface member. The sensing region may be provided to cover the entire circumference or an angle of at least 360° of the sensing surface. The sensing surface may be a surface which extends along an exterior operation surface of the user interface member, particularly on the interior of the user interface member or the user interface member body. The sensing surface may have an angular extension of 360°. The exterior operation surface may be a setting surface which has to be manipulated by the user, e.g. rotated, in order to perform a dose setting operation with the user interface member. The angular or circumferential disposition of the sensing surface may be relative to the rotation axis for dose setting.

In one embodiment, the sensor electrode system is configured such that one reference electrode portion and one sensing electrode portion can be or are disposed at opposite locations, particularly along the sensing surface and/or when the electrode system is provided in the electronic system, e.g. such that the sensing region conforms to the sensing surface. Thus, when arranged in the electronic system, one sensing electrode portion and one reference electrode portion may be offset relative to one another by 180°. Both portions may face towards the sensing surface. Every sensing electrode portion may have an oppositely disposed or associated reference electrode portion, e.g. offset by 180° from that portion. It has been discovered that providing the sensing electrode portion and the reference electrode portion at opposite locations relative to each other may result in particularly pronounced variations perceivable by the sensor controller caused by the user's finger(s) for or during dose setting as it is very likely that the user touches the user interface member at opposite locations with the thumb and another finger such as the index finger for the dose setting operation. It is particularly advantageous, if the number of sensing electrode portions and reference electrode portions is odd for this purpose.

In one embodiment, one of the reference electrode and the sensing electrode, e.g. the reference electrode or the sensing electrode, in the sensing region passes an end of the other one of the sensing electrode and the reference electrode, e.g. the sensing electrode or the reference electrode, respectively. Alternatively or additionally, the other one of the sensing electrode and the reference electrode may be arranged in between two portions of the one of the reference electrode and the sensing electrode. The two portions of the one of the reference electrode and the sensing electrode may run or be oriented parallel to each other, e.g. at least along the majority of the other one of the sensing electrode and the reference electrode, and/or may be connected by an electrode portion which passes the end of the one of the sensing electrode and the reference electrode.

In one embodiment, the sensing electrode is a first sensing electrode and the sensing region is a first sensing region. One of the electrically conductive electrode tracks of the electrode arrangement may form, may define or may be a second sensing electrode. The second sensing electrode may extend in a second sensing region of the flexible conductor carrier. The second sensing region and the first sensing region may be separated from one another, e.g. non-overlapping. The second sensing region and the first sensing region may be connected by a connection region of the flexible conductor carrier. The electrode track for the second sensing electrode may extend within the connection region such that it can define the second sensing electrode in the second sensing region. As opposed to the first sensing region and/or the second sensing region, the connection region may have a smaller dimension, e.g. a smaller length and/or smaller width. The conductive electrode track for the second sensing electrode is expediently electrically separated or insulated from the electrode track for the first sensing electrode on the flexible conductor carrier. The electrode track for the second sensing electrode may be electrically separated or insulated from the electrode track for the reference electrode.

In one embodiment, the flexible conductor carrier is configured such that the first sensing region and the second sensing region are movable relative to one another. The sensing regions may be movable relative to one another such that the surfaces with the sensing electrodes, i.e. the first sensing electrode and the second sensing electrode, face in different directions. For example, the first sensing region and the second sensing region may be configured such that they can be arranged relative to one another such that the second sensing region and the first sensing region are perpendicular relative to one another, i.e. the normal vectors of the surfaces of the flexible conductor carrier may be perpendicular in the sensing regions. One of the sensing regions may have an essentially plane surface and the other sensing region may have a curved surface or angularly oriented surface which faces in the radial direction, when the electrode system is provided in the electronic system.

In one embodiment, the first sensing region and the second sensing region are configured to be assigned or are assignable to exterior operation surfaces which face in different directions. For example, the first sensing electrode may be assigned to a setting surface of a user interface member. Alternatively or additionally the second sensing electrode may be assigned to a delivery surface of the user interface member.

In one embodiment, the reference electrode or the conductive electrode track therefor extends along the first sensing electrode in the first sensing region and/or along the second sensing electrode in the second sensing region. That is to say, the first sensing electrode and the second sensing electrode may have a common reference electrode. Thus, one conductive electrode track can be used for the reference electrode in two different sensing regions.

In one embodiment, the reference electrode or the conductive electrode track therefor is arranged between the first sensing electrode or the electrode track therefor and the second sensing electrode or the conductive electrode track therefor. Thus, when the first and second sensing electrodes or the electrode tracks therefor extend along each other, they are still separated by the reference electrode. This configuration makes electrical separation between the first and second sensing electrodes particularly easy and reliable and may at least reduce the likelihood of crosstalk between sensing electrodes.

In one embodiment, the reference electrode or the associated electrode track surrounds the sensing electrode or the associated electrode track in the sensing region. The reference electrode, e.g. in the sensing region, may have a first portion and a second portion. Both portions may be oriented along a main direction of extension of the flexible conductor carrier or the sensing region. The portions may run parallel to one another. The sensing electrode may be arranged, e.g. confined between the two portions of the reference electrode. The first and second portions may be connected by a transition portion. The transition portion may pass an end of the sensing electrode, when extending from the first portion to the second portion. The end may be remote from the contact connection region (see below).

In one embodiment, the first sensing electrode and the second sensing electrode are configured to be provided with the same or different electrical potentials during operation of the electronic system.

In one embodiment, the connection region between the first and second sensing regions has a width which is less than the width of the first sensing region and/or less than the width of the second sensing region. The width of the second sensing region may be less than the width of the first sensing region. Having a connection region with a small width facilitates the movable connection between the first and second sensing regions. The electrode tracks for the reference electrode and the second sensing electrode may extend along the connection region. The electrode tracks may be deformed together with the connection region.

In one embodiment, the flexible conductor carrier comprises a contact connection region. The contact connection region may be a region which is outside of the connection region, the connector region (see further below) and/or the sensing region(s). The contact connection region may be provided to electrically conductively connect the respective electrode track or the associated electrode, such as the (first) sensing electrode, the reference electrode, and/or the second sensing electrode to another component of the electronic system such as the sensor controller. The sensor controller may have a plurality of channels, preferably corresponding in number to the number of electrodes or electrode tracks provided on the electrode system. For example, the sensor controller may have one channel if only one electrode is provided, two channels if two electrodes are provided, such as the sensing electrode and the reference electrode, or three channels if three electrodes are provided such as the first sensing electrode, the second sensing electrode and the reference electrode. Just for completeness it should be mentioned that instead of having a common reference electrode, also separate reference electrodes could be provided for the first and second sensing electrodes. In this case, the sensor controller may have four channels. The contact connection region may be movable, e.g. foldable, relative to the sensing region(s). The contact connection region may be provided to electrically conductively connect the electrode tracks on the flexible conductor carrier with the sensor controller and/or with an other conductor carrier. The sensor controller may be arranged on an other, e.g. rigid, conductor carrier. The contact connection region may be provided to be connected with a connector, e.g. an fcc connector or a slimstack connector. The first sensing region may be arranged between the contact connection region and the second sensing region or between the contact connection region and the connector region as seen along the extension of the flexible conductor carrier (e.g. from the contact connection region to the second sensing region or the connector region respectively). Alternatively, the second sensing region or the connector region may be arranged between the contact connection region and the first sensing region.

In one embodiment, the reference electrode, the (first) sensing electrode and/or the second sensing electrode or the electrode tracks therefor extend from the associated sensing region into the contact connection region and/or can be contacted electrically in the contact connection region. The respective electrode may be accessible for electrical contact connection in the contact connection region. Terminals for the respective electrode (or electrode track) may be provided in the contact connection region.

In one embodiment, the sensing region is configured to cover at least 270°, at least 300°, at least 350° of the angular extension of a circumferentially disposed surface of the user interface member, e.g. the sensing surface mentioned above and/or when conforming to that surface. The flexible conductor carrier may be configured to cover more than 360° of the extension of the surface. In this manner, via the conductor carrier, particularly sensible regions of the flexible conductor carrier may be shielded from an outer surface. That is to say, the flexible conductor carrier may cover one or more sensible regions of the flexible conductor carrier, e.g. the connection region and/or the contact connection region.

In one embodiment, the electrode system is a flexible printed circuit board.

In one embodiment, the electrode arrangement is arranged in the interior of the user interface member, that is to say inaccessible from the exterior.

In one embodiment, one of the electrically conductive electrode tracks may form, may define or may be a connector electrode. The connector electrode may extend in a connector region of the flexible conductor carrier. The connector electrode or the connector region may be provided for electrically connecting a further electrode, e.g. a separate sensing electrode, to the electrode arrangement. The further electrode may be separate from the electrode arrangement, e.g. a separate part. The further electrode may be a metal part. The connector electrode may be exposed in the connector region of the flexible conductor carrier to enable mechanical contact of the connector electrode with the further electrode. In the electronic system, the further electrode may be arranged between the flexible conductor carrier and the exterior operation surface, which may be the delivery surface. The further electrode may be conductively connected to a power supply via the electrode arrangement. The further electrode may be elastically deformable. The further electrode may act as a biasing member (see further below) and/or may be arranged to bias one or more elements or components of the electronic system away from the exterior operation surface, e.g. a portion of the flexible conductor carrier. The connector region may be arranged in a region of the conductor carrier which is or is to be associated with the delivery surface. The further electrode may take over the role of the (second) sensing region or (second) sensing electrode (or the sensing region/electrode associated with the delivery surface). Hence, features which are described above and below for the (second) sensing region or electrode (or the sensing region associated with the delivery surface) do also apply for the connector region and vice versa. The first sensing region (or the one associated with the setting surface) is expediently provided in addition to the connector region and the further electrode.

In one embodiment, the electrode system, e.g. the electrode arrangement, comprises a terminal portion which is configured to be, e.g. directly, connected to a power supply. The terminal portion may be a portion of the electrode track forming the reference electrode or the sensing electrode, for example. The terminal portion may be accessible on a side of the flexible conductor carrier facing away from the exterior operation surface, for example. The biasing member (see further above or below), e.g. the further electrode, may be arranged to bias the terminal portion towards, e.g. into mechanical contact with, a terminal of a power supply. The terminal portion may be arranged on a side of the flexible conductor carrier remote or opposite from the connector electrode in the connector region. The terminal portion may be electrically insulated from the connector electrode.

In one embodiment, a reference electrode portion extends along the connector electrode, e.g. along an exterior or outer edge of the connector electrode, in the connector region. Having one or more portions of the reference electrode associated with the connector electrode results in the reference electrode being close to the further electrode once the electronic system has been assembled. Having the reference close to the further electrode is advantageous even if the electrode arrangement itself does not provide the sensing electrode but the further electrode does.

In one embodiment, as seen in top or plan view onto the conductor carrier, e.g. when the carrier is in a flat configuration, the connector electrode is arranged between the reference electrode portion and the terminal portion.

In one embodiment, the electronic system comprises a user interface member. The user interface member has at least one exterior operation surface. The exterior operation surface may be arranged to be manipulated by the user for a dose operation. The dose operation may be a dose setting operation to set a dose to be delivered by a drug delivery device and/or a dose delivery operation to deliver a dose, e.g. the dose which has been previously set. For manipulating the user interface member the exterior operation surface may have to be touched by the user. The exterior operation surface may be a setting surface for the dose setting operation, e.g. a surface facing in the radial direction and/or circumferentially disposed, or a delivery surface, such as a surface facing in an axial, e.g. in the proximal, direction. The user interface member may have to be moved for the dose setting operation, e.g. rotationally. The amount of rotation may be proportional to the size of the set dose. The user interface member may have to be moved for the dose delivery operation, e.g. axially. The (respective) movement of the user interface member may be with respect to a housing, e.g. a housing of the drug delivery device or of a drug delivery device unit.

In one embodiment, the user interface member comprises a user interface member body. The user interface member body may form or define the exterior operation surface or surfaces. The body may have a cup-like configuration. The body may have a closed proximal end for the delivery surface. The distal end may be open, e.g. for the connection of the user interface member to a mechanism member of a dose setting and drive mechanism of or for the drug delivery device.

In one embodiment, the exterior operation surface and/or the user interface member body may be electrically insulating.

In one embodiment, the electronic system comprises a user proximity detection unit. The user proximity detection unit may be arranged in the interior of the user interface member, especially in the interior of the user interface member body. The user proximity detection unit may be arranged and configured to detect whether the user is close to or touches the exterior operation surface. When the user proximity detection unit has confirmed that the user is close to or touches the exterior operation surface, the user proximity detection unit may provide or trigger generation of an electrical signal, such as a use or proximity signal. The signal may be indicative that the user is close to or touches the exterior operation surface and/or that a dose operation, e.g. a dose setting operation or a dose delivery operation, is about to be commenced. The user proximity detection unit may comprise the electrode system as described above. The user proximity detection unit may further comprise an electronic sensor controller, e.g. a microcontroller or an ASIC. The sensor controller may be electrically conductively connected to the electrode system, e.g. as has been discussed further above already. The sensor controller may have different channels for the different tracks or electrodes. The signal may be provided by the user proximity detection unit in response to the user touching the exterior operation surface or the user being close to the exterior operation surface but not yet touching the exterior operation surface and being less than a predetermined distance away from the exterior operation surface. The predetermined distance may be less than or equal to one of the following values: 5 mm, 4 mm, 3 mm, 2 mm, 1 mm, 0.5 mm, 0.4 mm, 0.3 mm, 0.2 mm.

In one embodiment, the (first) sensing electrode is assigned to the setting surface of the user interface member, and/or the second sensing electrode is assigned to the delivery surface of the user interface member. Thus, via the electrode system, user proximity relative to different surfaces can be detected with the same electrode system.

In one embodiment, the first sensing region is associated with the setting surface. Alternatively or additionally, the second sensing region is associated with the delivery surface.

In one embodiment, the electronic system comprises a power supply, such as a rechargeable or non-rechargeable battery, e.g. comprising at least one coin cell. The power supply may provide power for the electrically operated components of the electronic system. The electronic system may be self-contained. That is to say, the power supply may not be exchangeable and, consequently permanently installed in the system. Thus, when the power contained in the power supply has been consumed, the electronic system may have to be disposed, if the supply is not rechargeable.

In one embodiment, the electronic system comprises an electronic control unit. The electronic control unit may be provided in addition to the sensor controller. In this way, the sensor controller may be used to switch the electronic control unit to a state of higher power consumption. The electronic control unit then, again, may switch one or more further units to a state of higher power consumption, e.g. from non-operational to operational. The further units may comprise a motion sensing unit and/or a communication unit. The motion sensing unit may be provided to capture dose related data during the dose delivery operation, e.g. characteristic for the size of the delivered dose. The communication unit may be provided to establish a communication channel to another device and/or to communicate dose related data to another device, e.g. a mobile phone or a personal computer, such as after the end of the dose delivery operation.

In one embodiment, the sensing region, e.g. the second sensing region and/or the first sensing region, is biased towards the exterior operation surface, e.g. by a resilient force or spring force. For example, the sensing region may be biased against an inner surface of the user interface member or user interface member body which is close to the exterior operation surface, e.g. arranged on that side of a wall of the user interface member body which faces away from the exterior operation surface, where the wall defines the exterior operation surface on the exterior. Hence, the sensing region may abut or conform to an interior surface of the user interface member or user interface member body.

In one embodiment, the sensing region, e.g. the first sensing region and/or the second sensing region, is biased towards the exterior operation surface by a force resulting from an elastic deformation of the electrode system or the flexible conductor carrier.

In one embodiment, the electronic system comprises a biasing member, e.g. a resilient member such as a spring. The biasing member may be arranged to bias a region of the flexible conductor carrier relative to the exterior operation surface and/or the user interface member body, e.g. towards or away from the user interface member body. As noted further above, the biasing member may be formed by the further electrode, which may act as separate sensing electrode. Hence, referrals to the biasing member, e.g. regarding its arrangement, may be understood as referrals to the further electrode, especially if a further electrode is provided as sensing electrode and the electrode is not implemented by the electrode arrangement.

In one embodiment, the sensing region, e.g. the second sensing region, is biased towards the exterior operation surface by the biasing member. In other words, the biasing member may be provided in the electronic system for mechanically biasing the sensing region towards the exterior operation surface.

In one embodiment, the biasing member is arranged between the user interface member body, and the connector portion e.g. between the exterior operation surface (such as the delivery surface) and the connector portion.

In one embodiment, the biasing member is electrically conductive. The biasing member may be electrically conductively connected to the power supply. The biasing member may be electrically conductively connected to the sensor controller and/or to other electronic or electrically powered units of components of the electronic system, e.g. the power supply.

The biasing member may serve for guiding electrical power from the power supply to other units or components of the electronic system, such as the sensor controller or the electronic control unit. The biasing member may bias the sensing region proximally relative to the user interface member body, e.g. into abutment against the inner surface of that section of the user interface member body which forms or defines the delivery surface. The sensing region may be clamped between the user interface member body which defines the exterior operation surface with which the sensing region is associated and the biasing member.

The biasing member may bias the flexible conductor carrier towards the power supply (e.g. relative to the user interface member body) and/or the power supply towards the other carrier (e.g. the rigid conductor carrier), optionally so as to ensure an electrical conductive connection between the power supply and the other carrier, e.g. a conductor or terminal thereof. The biasing member may serve as separate sensing electrode and be conductively connected with the connector electrode in the connector region of the flexible conductor carrier, e.g. by mechanical contact between connector electrode and biasing member.

In one embodiment, the flexible conductor carrier has a plurality of sensing regions associated with the same exterior operation surface, e.g. the delivery surface or the setting surface. In each sensing region, one or more electrode tracks of the electrode arrangement may extend. The configuration of the electrode tracks in the different sensing regions may be identical. Each sensing region, e.g. each second sensing region, may be connected to another carrier region of the flexible conductor carrier, e.g. the first sensing region, via a distinct connection region of the flexible conductor carrier. In this case, the exterior operation surface may be covered or monitored by a plurality of sensing regions which, preferably, have the same electrode track configuration and/or portions of the same electrode tracks provided in each sensing region.

Therefore, monitoring the exterior operation surface can be distributed over a variety of sensing regions. This facilitates reducing the size and weight of the respective sensing region. Accordingly, it is easier for the connection region on account of its intrinsic elasticity to bias the respective sensing region towards the exterior operation surface. In this case, it is easier to dispense with the separate biasing member for this purpose or to dimension the biasing member differently.

In one embodiment, the user proximity detection unit is configured to monitor proximity of an object (e.g. a conformable object such as the user's finger) to the exterior operation surface.

In one embodiment, the exterior operation surface comprises a surface structure. The surface structure may comprise one or more indentations or recesses. The indentations or recesses may be separated from one another or be connected. The indentations or recesses may be dimensioned such that a conformable object, such as the skin on the user's finger, e.g. the thumb and/or the index finger, may be deflected into the indentation or recess when the user touches the exterior operation surface. In this manner, the distance between the user's skin and the sensing region may be reduced. The closer the object to be sensed is to the sensing region the more pronounced is usually the signal. By means of the surface structure the risk that a non-conformable object, e.g. a metal object, is wrongly assessed as being the user close to the exterior operation surface can be reduced. The surface structure of the exterior operation surface may be defined by the user interface member body. The user interface member body may have a varying thickness. For the setting surface as exterior operation surface, the radial thickness of the body may vary along the axial extension and/or the angular extension of the setting surface. For the delivery surface as exterior operation surface, the axial thickness of the body may vary along the radial extension and/or the angular extension of the delivery surface. The thickness of the user interface member body, particularly in the region between the sensing region and the associated exterior operation surface, may define the distance between the sensing surface and the associated exterior operation surface. Thus, this distance may vary as well, e.g. due to the surface structure.

In one embodiment, the exterior operation surface is configured such that the user proximity detection unit has a higher sensitivity for softer or more flexible conductive objects (e.g. the users skin on fingers or the thumb) that conform to the exterior operation surface easier as compared to harder or stiffer conductive objects (e.g. metal and/or rigid objects) that do not conform as easily to the exterior operation surface. This may be achieved by the surface structure as set forth further above.

In one embodiment, the sensor electrode system is configured such that the electrode arrangement, preferably in the sensing region, is adjusted to, preferably configured to compensate for, thickness variations in the user interface member body along the exterior operation surface and/or distance variations of the exterior operation surface from the sensing region associated with the exterior operation surface. The thickness or distance variations may be due to the surface structure of the user interface member body. The electrode arrangement may be configured such that a sensitivity of a sensor comprising the sensor electrode system is homogenous along the exterior operation surface, e.g. the setting surface, which is monitored via the sensor electrode system, in particular despite the thickness variations. Hence, variations in the sensor sensitivity along the exterior operation surface, e.g. the circumferential direction and/or the axial direction along the setting surface, may be smaller for the proposed solution than the sensitivity variations along an exterior reference surface monitored by the same sensor. The reference surface may be smooth and, hence, have a constant distance from the sensing region of the sensor electrode system conforming to the inner or sensing surface of the user interface member body. The general shape of the reference body may be the same as the one of the user interface member body but without thickness variations. The reference surface is expediently a surface of a reference body of the same material as the user interface member body. The reference body thickness along the reference surface may be constant and may be between the maximum thickness and the minimum thickness of the user interface member body. The distance of the exterior reference surface from the sensing region may be between the maximum and the minimum distance of the exterior operation surface from the sensing region. The electrode arrangement may be adjusted to the thickness variations by the configuration of the electrode track or tracks in the sensing region. For example, the reference electrode and the sensing electrode may be interleaved in the sensing region which is considered to be particularly suitable for providing a homogenous sensitivity even if a structured surface is monitored by the sensor electrode system.

In one embodiment, a sensor, e.g. a capacitive sensor, comprises the sensor controller and the sensor electrode system.

In one embodiment, in the electronic system, the contact connection region and the second sensing region are oriented in the same direction or the contact connection region and the connector region are oriented in the same direction.

In one embodiment, in the electronic system, the contact connection region and/or the second sensing region are oblique or perpendicular, relative to the first sensing region.

In one embodiment, the electronic system comprises a carrier, such as a conductor carrier, e.g. a rigid conductor carrier. The carrier may be provided in addition to the flexible conductor carrier of the electrode system. The sensor controller, the electronic control unit and/or further units of the electronic system, such as the communication unit and/or the motion sensing unit, may be arranged on the carrier and/or conductively connected to conductors provided on the carrier. The carrier may be a printed circuit board.

In one embodiment, the user interface member comprises a user interface member part. The user interface member part may be, preferably axially and/or rotationally, fixed to the user interface member body. An interior of the user interface member may be delimited by the user interface member body and the user interface member part. The interior may be sealed, e.g. against moisture and/or dirt. The user interface member part may be received in the distal opening of the user interface member and, preferably, close that opening. The user interface member part may be a chassis.

In one embodiment, the carrier is secured, e.g. axially and/or rotationally, to the user interface member part.

In one embodiment, the electrode system, e.g. the sensing region, is wrapped around the user interface member part.

In one embodiment, the sensing region extends along an outer surface, e.g. a radially facing outer surface, of the user interface member part and/or the carrier. The sensing region may fully or partially surround the carrier or the user interface member part, especially in the angular or circumferential direction, e.g. relative to an axis parallel to the distal direction.

In one embodiment, the contact connection region of the electrode system extends inwardly relative to the outer surface of the user interface member part, e.g. towards the (rigid) carrier. The contact connection region may be conductively connected to the sensor controller e.g. directly or via conductors on the carrier.

In one embodiment, the flexible conductor carrier is fixed or secured to the user interface member part, e.g. rotationally and/or axially. The sensing region may be fixed to the user interface member part, e.g. an outer surface thereof. Protrusions of the user interface member part may be received in cutouts of the flexible conductor carrier or the electrode system for fixing the flexible conductor carrier to the user interface member part, for example.

In one embodiment, the power supply is arranged between a portion of the flexible conductor carrier and the (rigid) carrier. The portion of the flexible conductor carrier may be the connector region. A connection region of the flexible conductor carrier, e.g. the connection region extending towards the contact connection region, may extend from the portion of the flexible conductor and/or the side of the power supply which is remote from the carrier towards the carrier. The contact connection region may be mechanically and/or electrically connected to the carrier (e.g. to a conductor or a terminal on the carrier). The contact connection region may conductively connect the flexible conductor carrier to the carrier and/or the sensor controller. The power supply may be secured or fixed to the user interface member part and/or to the carrier by the flexible conductor carrier, e.g. via the associated connections to the carrier on the one hand and to the user interface member part on the other hand. In other words, in the present disclosure, the flexible conductor carrier may serve as a retainer or securing member to secure components of a subassembly for the electronic system relative to one another. The subassembly may be inserted into the user interface member body. The components may be or may comprise: the carrier, the user interface member part, and/or the power supply. In addition to these components, the subassembly may comprise the flexible conductor carrier.

In one embodiment, in the method of manufacturing or assembling the electronic system, an electrode system for a drug delivery device is provided. The electrode system may be the one discussed above. The electrode system may comprise a flexible conductor carrier which is preferably electrically insulating. The electrode system may further comprise an electrode arrangement. The electrode arrangement may comprise at least one electrically conductive electrode track. The electrode track may extend along the flexible conductor carrier. Also, in the method, a part, e.g. the user interface member part, may be provided.

In one embodiment, an electronic unit, e.g. the sensor controller, may be conductively connected to the electrode arrangement, e.g. to the conductive electrode track.

In one embodiment, the method comprises deforming the electrode system, e.g. by folding one or more portions relative to other portions of the electrode system. The electrode arrangement may be deformed such that a surface of the conductor carrier extends along or conforms to the part, e.g. an outer surface of the part. The surface of the flexible conductor carrier facing towards the part may be the surface of the flexible conductor carrier which is remote from the electrode arrangement. The opposite side may define the sensing region. Deforming the electrode system or the flexible conductor carrier may occur before or after connecting the electrode track conductively with the electronic unit, e.g. via the contact connection region of the flexible conductor carrier.

In one embodiment, before the electrode system is deformed to extend along the outer surface of the part, the remainder of the flexible conductor carrier is deflected relative to the contact connection region which may be connected to the electronic unit already.

In one embodiment, after connecting the electrode track conductively with the electronic unit and/or after deforming the flexible conductor carrier to conform to the part, the user interface member body may be guided over the surface of the flexible conductor carrier facing away from the part. Hence, in the electronic system, a region of the flexible conductor carrier may be arranged between an inner surface of the user interface member body and an outer surface of the (user interface member) part. That region may be or comprise the sensing region, e.g. the first sensing region, as discussed further above. The user interface member body and the user interface member part may be connected to one another, e.g. rigidly.

In one embodiment, the method further comprises providing a biasing member and arranging the biasing member and a region of the flexible conductor carrier, e.g. a sensing region, such as the second sensing region, such that the biasing member is arranged to bias the sensing region away from the part and/or towards an inner surface of the user interface member body. The inner surface may be a surface of the user interface member facing away from the exterior operation surface, e.g. an inner surface of a wall of the user interface member body which defines the delivery surface on the exterior.

In one embodiment, a subassembly is provided. The subassembly may be provided to be rotationally and/or axially secured to the part, e.g. after the electrode track has been connected to the electronic unit. The carrier, the power supply, the electronic unit, and/or the biasing member may belong to the subassembly.

In one embodiment, the subassembly comprises an orientation feature. The orientation feature may be provided to ensure that the subassembly can be arranged on or connected to the part in only a defined relative orientation, e.g. relative angular orientation. The defined relative orientation may comprise only one relative orientation. This ensures that electronic unit(s) of the subassembly are arranged relative to the part in defined positions.

In one embodiment, the flexible conductor carrier extends along the outer edges, particularly the radial outer edges of the power supply and/or the part, e.g. the user interface member part.

In one embodiment, the drug delivery device comprises a reservoir retainer for retaining a reservoir with drug, e.g. a cartridge, and/or the device comprises the reservoir. The reservoir may comprise drug sufficient for a plurality of, preferably user-settable doses, to be delivered by the drug delivery device.

In one embodiment, the drug delivery device is a pen-type device and/or an injection device, e.g. a needle based injector.

In one embodiment, the electronic system is configured as a, preferably reusable, add-on for a drug delivery device unit. The system may be configured to be attached to the drug delivery device unit. That is to say, the electronic system may be configured to be used with a plurality of drug delivery device units. The respective drug delivery device unit may be a disposable drug delivery device unit and/or the respective drug delivery device unit may be fully operational for performing dose setting operations and dose delivery operations. The drug delivery device unit may comprise the reservoir. The drug delivery device unit may be free of electrically operated units or components for such units.

In one embodiment, the drug delivery device or the drug delivery device unit comprises a dose setting and drive mechanism. The dose setting and drive mechanism may comprise a first member which is connected or connectable to the electronic system. The dose setting and drive mechanism may comprise a second member. First and second member may be coupled for a dose setting operation, e.g. rotationally locked. For the dose delivery operation, the first and second member may be decoupled, e.g. such that relative rotational movement is allowed. The dose setting and drive mechanism may further comprise a piston rod. The first member may be coupled, e.g. directly and/or threadedly, to the piston rod. During the dose delivery operation, relative rotational movement between the first and second members may be measured by the electronic system to determine the size of the delivered dose.

In one embodiment, a kit for a drug delivery device comprises the drug delivery device unit and the electronic system. The system may be attachable to the device unit to form the drug delivery device. Features disclosed above and below for the drug delivery device, especially the ones that are not directly related to the electronic system, should also apply for the drug delivery device unit and vice versa.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end, where a needle unit is or is to be mounted to the device, for example.

In an advantageous embodiment, a sensor electrode system for a drug delivery device comprises:
a flexible conductor carrier which is electrically insulating; and
an electrode arrangement, wherein the electrode arrangement comprises at least two electrically conductive electrode tracks, wherein the electrically conductive electrode tracks extend along the flexible conductor carrier, wherein the electrically conductive electrode tracks are electrically separated from one another along the conductor carrier, wherein one of the electrically conductive electrode tracks forms a sensing electrode, and wherein the sensing electrode extends in a sensing region of the flexible conductor carrier.

In an advantageous embodiment, a method of manufacturing an electronic system for a drug delivery device comprises the following steps:
providing an electrode system, e.g. the electrode system discussed above, wherein the electrode system comprises a flexible conductor carrier which is electrically insulating and an electrode arrangement, wherein the electrode arrangement comprises at least one electrically conductive electrode track extending along the flexible conductor carrier,
providing a part, e.g. the user interface member part,
deforming the electrode system such that a surface of the flexible conductor carrier extends along the part, e.g. along an outer surface of the part; and
connecting the at least one electrically conductive electrode track conductively with an electronic unit, e.g. to form the electronic unit.

Features, which are disclosed in conjunction with different aspects and embodiments may be combined with one another even if such a combination is not explicitly discussed. For example, features relating to the electrode system, the electronic system may also apply for the device or the method or the kit and vice versa.

Further aspects, embodiments and advantages will become apparent from the following description of the exemplary embodiments in conjunction with the drawings.

### BRIEF DESCRIPTION OF THE FIGURES

FIG. 1 shows an embodiment of a drug delivery device.

FIG. 2 illustrates an embodiment of the electronic system.

FIGS. 3A to 3C illustrate an embodiment of the electronic system.

FIG. 4 illustrates an embodiment of the electrode system.

FIG. 5 illustrates an embodiment of the electronic system.

FIG. 6 illustrates an embodiment of the electrode system.

FIG. 7 illustrates an embodiment of the electronic system.

FIGS. 8A to 8C illustrate an embodiment of the electrode system.

FIGS. 9A to 9C illustrate an embodiment of the electronic system.

FIGS. 10A to 10D illustrate an embodiment of the electronic system and a method for assembling or manufacturing the electronic system.

FIG. 11 illustrates an embodiment of the electrode system.

FIG. 12 illustrates an embodiment of the electrode system.

FIG. 13 illustrates an embodiment of the electrode system.

FIG. 14 illustrates an embodiment of the electrode system.

FIGS. 15A to 15F illustrate an embodiment of the electrode system, of the electronic system and of the method.

### DETAILED DESCRIPTION

In the figures, identical elements, identically acting elements or elements of the same kind may be provided with the same reference numerals.

In the following, some concepts will be described with reference to an insulin injection device. The systems described herein may be implemented in this device. The present disclosure is however not limited to such an application and may equally well be used for or in injection devices that are configured to eject other medicaments or drug delivery devices in general, preferably pen-type devices and/or injection devices.

In the following, embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein may include power management techniques (e.g. to facilitate small batteries and/or to enable efficient power usage) or related concepts.

Certain embodiments in this document are illustrated with respect to an injection device where an injection button and grip (dose setting member or dose setter) are combined e.g. similar to Sanofi's ALLSTAR® device. The injection button may provide the user interface member for initiating and/or performing a dose delivery operation of the drug delivery device. The grip or knob may provide the user interface member for initiating and/or performing a dose setting operation. The devices may be of the dial extension type, i.e. their length increases during dose setting. Other injection devices with the same kinematical behaviour of the dial extension and button during dose setting and dose expelling operational mode are known as, for example, the Kwikpen® or Savvio® device marketed by Eli Lilly and the FlexPen®, FlexTouch® or Novopen® device marketed by Novo Nordisk. An application of the general principles to these devices therefore appears straightforward and further explanations will be omitted. However, the general principles of the present disclosure are not limited to that kinematical behaviour. Certain other embodiments may be conceived for application to injection devices where there are separate injection button and grip components/dose setting members e.g. Sanofi's SoloSTAR®. Thus, the present disclosure also relates to systems with two separate user interface members, one for the dose setting operation and one for the dose delivery operation. In order to switch between a dose setting configuration of the device and a dose delivery configuration, the user interface member for dose delivery may be moved relative to the user interface member for dose setting. If one user interface member is provided, the user interface member may be moved distally relative to a housing. In the course of the respective movement, a clutch between two members of the dose setting and drive mechanism of the device changes its state, e.g. from engaged to released or vice versa. When the clutch, e.g. formed by sets of meshing teeth on the two members, is engaged, the two members may be rotationally locked to one another and when the clutch is disengaged or released, one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

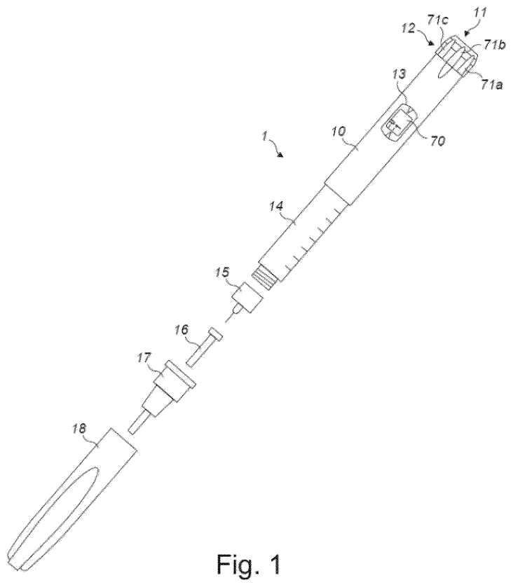

The injection device 1 of FIG. 1 is an injection pen that comprises a housing 10 and contains a container 14, e.g. an insulin container, or a receptacle for such a container. The container may contain a drug, e.g. insulin. The container may be a cartridge or a receptacle for a cartridge which may contain the cartridge or be configured to receive the cartridge. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialled in’ by turning a dosage knob 12, and a currently programmed or set dose is then displayed via dosage window 13, for instance in multiples of units. The units may be determined by the dose setting mechanism which may permit relative rotation of the knob 12 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a data collection device or electronic system. An electronic system which may be attachable to the user interface member (knob 12 and/or button 11) or, in general, to elements or members of a dose setting and drive mechanism of the drug delivery device 1 will be described in more detail below. The electronic system may be provided within the user interface member, for example. The electronic system which will be described in more detail below can also be configured as an add-on for a drug delivery device.

The injection device 1 may be configured so that turning the dosage knob 12 causes a mechanical click sound to provide acoustical feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display window 13 will be ejected from injection device 1. When the needle 15 of injection device 1 remains for a certain time in the skin portion after the dosage knob 12 is pushed home, the dose is injected into the patient's body. Ejection of the insulin dose may also cause a mechanical click sound, which is however different from the sounds produced when rotating the dosage knob 12 during dialing of the dose.

In this embodiment, during delivery of the insulin dose, the dosage knob 12 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached.

Furthermore, before using injection device 1 for the first time, it may be necessary to perform a so-called “prime shot” to ensure fluid is flowing correctly from insulin container 14 and needle 15, for instance by selecting two units of insulin and pressing dosage knob 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

As explained above, the dosage knob 12 also functions as an injection button 11 so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting the user interface member is moved proximally and/or rotates relative to the housing. During dose delivery, the user interface member moves axially, e.g. distally, preferably without rotating relative to the housing or body.

In the following, an embodiment for an electronic system for a drug delivery device is disclosed.

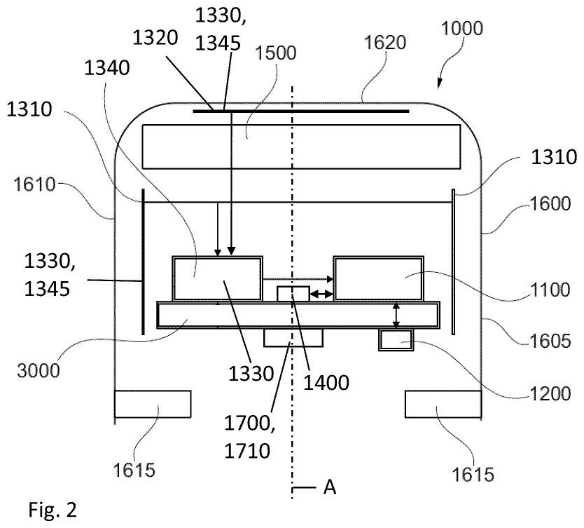

FIG. 2 schematically illustrates an embodiment of an electronic system 1000. The system 1000 comprises a user interface member 1600. The user interface member is designed to be operated during a dose setting operation and/or a dose delivery operation by the user. The user interface member 1600 has different exterior operation surfaces. The operation surfaces may be defined by exterior surfaces which are accessible from the exterior of a user interface member body or housing 1605. The user interface member 1600 has a setting surface 1610 which is arranged to be gripped by the user for the dose setting operation. The dose setting operation, especially if performed by rotation, usually requires two points of contact with the user interface member. For example, the user usually grips the user interface member with two fingers such as the index finger and the thumb. The setting surface is a surface which circumferentially delimits the user interface member 1600, e.g. a radially facing surface. The user interface member 1600 also has a delivery surface 1620. The delivery surface is arranged to be contacted, e.g. pressed, by the user for dose delivery. The delivery surface 1620 is an axially facing surface, e.g. a proximally facing surface. In the depicted embodiment, the user interface member 1600 has a cup-like configuration. As noted above, embodiments of the disclosure can employ different user interface members for setting and delivery. The dose setting operation may involve rotation of the user interface member 1600 around its central axis which may run perpendicular to the delivery surface 1620 and through the delivery surface (not explicitly shown).

Within the user interface member 1600, e.g. within an interior hollow defined by the user interface member body 1605, some additional elements or units of the electronic system are housed or arranged. Specifically, the system comprises an electrical user proximity detection unit 1330. The user proximity detection unit 1330 is configured to detect whether the user's finger (e.g. index finger and/or thumb) is close to or touches the setting surface 1610 and/or close to or touches the delivery surface 1620. The user proximity detection unit 1330 comprises a sensor controller 1340, e.g. a controller of the type Azoteq IQS 228-AS. The sensor controller 1340 is operatively, e.g. electrically conductively, connected to an electrode system 1345 with an electrode arrangement. The sensor controller and the sensor electrode system together form a capacitive sensor. The sensor electrode system 1345 provides or comprises a setting sensing region 1310 (first sensing region). The sensor electrode system 1345 provides or comprises a delivery sensing region 1320 (second sensing region). The setting sensing region 1310 is associated with the setting surface 1610. The setting sensing region 1310 expediently extends along at least the majority or the entire inner surface of the user interface member body 1605 which faces away from the setting surface, e.g. at least along 350° or 360° of the angular extension of the inner surface. The setting sensing region 1310 faces towards the setting surface 1610. The delivery sensing region 1320 is associated with the delivery surface 1620.

The delivery sensing region 1320 faces towards the delivery surface 1620. Sensing region 1320 may be plane. Sensing region 1310 may have a curved, e.g. cylindrical configuration, which encircles or extends around the central or main axis A of the user interface member. Instead of having a setting sensing region and a delivery sensing region, only one of these regions may be provided, i.e. the setting sensing region or the delivery sensing region. Also, in the depicted embodiment both sensing regions or the associated electrodes are connected to a common sensor controller 1340. It is also conceivable that the setting sensing region and the delivery sensing region are connected to separate sensor controllers. However, it is expedient to connect both sensing regions to the same sensor controller, preferably to different channels of the sensor controller. Suitable embodiments for the electrode system 1345 are described later on in more detail. The sensor controller 1340 may be a low-power controller that monitors input capacitance or the electrical field from the sensing region of the electrode system 1345, e.g. from the respective channel or from a combination of channels, and outputs a signal if the capacitance, a change in the capacitance or a change in the electrical field detected in the sensing region meets a predetermined criterion, e.g. when the monitored quantity or value rises above or falls below a threshold. The criterion is expediently chosen such that, if the criterion is met, this is characteristic for the user being close to or touching the respective exterior operation surface. The capacitive sensor can be configured to output the signal when the user is less than 0.5 mm, or 0.4 mm or 0.3 mm or 0.2 mm away from the monitored surface. We note, however, that the operating distance of the capacitive sensor can also be increased, if desired, or even decreased. The signal generated by the user proximity detection unit 1330 may be used to influence the operational state of the electronic system or a drug delivery device which comprises the system. Having setting and delivery sensing regions enables to distinguish which one of the exterior operation surfaces, i.e. setting surface or delivery surface, the user is close to. This can be used for different operations in the electronic system.

The electronic system 1000 further comprises an electronic control unit 1100. The control unit may comprise a processor, e.g. a microcontroller or an ASIC. Also, the control unit 1100 may comprise one, or a plurality of memory units, such as a program memory and/or a main memory. The program memory may be designed to store program code or software which when carried out by the electronic system controls operation of the system and/or the electronic control unit. The control unit 1100 is expediently designed to control operation of the electronic system 1000. The control unit 1100 may communicate via wired interfaces or wireless interfaces with further units of the electronic system 1000, e.g. the sensor controller 1340. It may transmit signals containing commands and/or data to the units and/or receive signals and/or data from the respective unit. The connections between the units and the electronic control unit are symbolized by the lines or arrows in FIG. 2. However, there also may be connections between the units, which are not illustrated explicitly. Likewise, the operative or conductive connection between the electrode system 1345 and the sensor controller 1340 is illustrated by arrows.

The electronic system further comprises a power supply 1500, such as battery, e.g. a coin cell, or a stack of multiple coin cells. The power supply may be configured to provide a total charge of approx. 50-500 mAh at a voltage of approx. 1.4-3V. The power supply 1500 is expediently conductively connected to the sensor controller 1340 and/or the electronic control unit 1100. The power supply may provide the entire power for the operation of the electronic system, preferably throughout its entire lifetime. Once the power remaining in the power supply is insufficient for operating the system, the system may have to be disposed of.

The electronic system 1000 further comprises a conductor carrier 3000, e.g. a (printed) circuit board. Components of electrical or electronic units of the electronic system or entire units may be arranged and/or conductively connected to conductors on the conductor carrier 3000. The conductor carrier 3000 is rigid. That is to say, the conductor carrier expediently keeps its shape under its own weight in any orientation relative to the ground and, preferably, even if forces are exerted onto the conductor carrier. The conductor carrier 3000 may be more rigid than a flexible conductor carrier 1350 of the sensor electrode system 1345 which is further discussed below. The flexible conductor carrier may or may not keep its shape under its own weight but be in any case deformable when external forces are applied. The conductor carrier 3000 may be fixed relative to the user interface member body 1605, either directly to the body or to another part which then may be fixed to the body (not explicitly shown, see the user interface member part or chassis 1670 discussed below). The sensor controller 1340 and the electronic control unit 1100 are arranged on and, preferably, conductively connected to conductors on the conductor carrier 3000. The power supply 1500 is arranged between the delivery surface 1620 and the conductor carrier 3000. This facilitates a compact design of the user interface member 1600.

The electronic system 1000 further comprises an electrical motion sensing unit 1200. The motion sensing unit 1200 may comprise one sensor e.g. only one sensor, or a plurality of sensors. The motion sensing unit is expediently designed to generate motion signals, such as electrical signals, which are indicative for movement of one member of the electronic system or the drug delivery device relative to another member—e.g. movement of the dial sleeve or number sleeve relative to the drive sleeve or button/knob in the device discussed further above in conjunction with FIG. 1—where the sensor may be fixedly connected to one of the members, e.g. to the knob or button or drive sleeve. The movement is expediently relative rotational movement. The relative rotational movement expediently occurs during the dose delivery operation e.g. only during the delivery operation. The respective sensor can be an optoelectronic sensor. The optoelectronic sensor may sense radiation emerging from a member moving relative to the sensor and impinging on the sensor to excite sensor signals or motion signals in the sensor, e.g. an optical encoder component. The radiation may be radiation reflected by the moving member and impinging on the member from a radiation source, such as an optoelectronic radiation source, e.g. an LED. The radiation source may be an IR source (IR-LED, an InfraRed Light Emitting Diode). The radiation source may be part of the sensor arrangement comprising the at least one sensor. One possible embodiment of the sensor is an IR-sensor which is configured to detect infrared light. The light source and the sensor may be arranged on the same component or member. The general functionality of optoelectronic sensor arrangements suitable for the electronic system discussed herein is disclosed in WO 2019/101962 A1, where the entire disclosure content is explicitly incorporated herein by reference for all purposes, especially as regards the different sensor arrangements and configurations. However, it should be noted that other sensor arrangements, e.g. using magnetic sensors, could be employed as well. In a motion sensing unit which has an electrically operated sensor and/or an electrically operated source for stimulating the sensor—such as a radiation emitter and an associated sensor—the power consumption may be particularly high and, hence, appropriate power management of electrical power available for powering the system may have a particular impact. For example, the signal generated by the user proximity detection unit may be used to switch the motion sensing unit from a non-operational state or off state into an operational state or on state. The electronic control unit 1100 may switch the motion sensing unit to the operational state of higher power consumption in response to the signal generated by the user proximity detection unit which may be fed to or detected by the electronic control unit 1100. The motion sensing unit, in the depicted embodiment, is arranged on a surface of the conductor carrier facing away from the delivery surface 1620 and/or the electronic control unit 1100 or from the sensor controller 1340. This is particularly suitable if an optoelectronic unit is used for motion sensing and the member the motion of which should be sensed is arranged on the same side of the conductor carrier as the motion sensing unit 1200.

The motion sensing unit 1200 may be designed to detect and preferably measure or quantify relative movement of one member of a dose setting and drive mechanism of or for the drug delivery device relative to another member of the dose setting and drive mechanism or relative to the housing 10 during a dose delivery operation. For example, the motion sensing unit may measure or detect relative rotational movement of two movable members of the dose setting and drive mechanism of the drug delivery device with respect to one another, e.g. movement of the dial sleeve a number sleeve relative to the drive sleeve or the user interface member button/knob. Based on movement data received from or calculated from the signals of the unit 1200, the electronic system, e.g. the control unit 1100, can calculate dose data, e.g. data on the currently delivered dose during the ongoing or completed dose delivery operation. The motion sensing unit 1200 is expediently configured to quantify the relative movement between a first member and a second member of the electronic system or the drug delivery device. The relative movement may be indicative for the delivered dose. The relative movement may be relative rotational movement. For example, the first member may rotate relative to the second member, such as during dose delivery. The motion sensing unit is expediently suitable to quantify the relative movement in whole-number multiples of one unit setting increment. The unit increment may be or may be defined by an angle greater than or equal to one of the following values: 5°, 10°. The unit setting increment may be or may be defined by an angle less than or equal to one of the following values: 25°, 20°. The unit setting increment may be between 5° and 25°, for example. The unit setting increment may correspond to a relative rotation of 15°, for example. The unit setting increment may be the rotation required to set the smallest settable dose to be delivered by the device. As has been explained above, the amount or distance of the relative movement determined by the motion sensing unit between the first and second members can be characteristic for the currently set dose in a dose setting operation or for the currently dispensed dose in a dose delivery operation. The size of the dose delivered may be determined by or correspond to the distance by which a piston rod of the dose setting and drive mechanism is displaced distally relative to the housing 10 during the dose delivery operation.

The motion sensing unit is preferably bidirectionally conductively connected to the electronic control unit 1100 as hinted by the double arrow. One direction may be the one where an activation signal is transmitted from the electronic control unit to the motion sensing unit to switch the unit to the operational state. In the other direction, motion signals may be sent from the motion sensing unit to the control unit, which may process the signals further, e.g. to calculate dose information or data.

The user interface member body 1605 is arranged between the electrode system 1345 and the exterior surface of the user interface member such that the user does not touch the electrode 1345 when touching the exterior surface in the region overlapping with the electrode 1345. It is, however, also conceivable that the user can touch the electrode. Having the electrode in the interior facilitates sealing the interior against external influences, e.g. moisture and/or dirt. Having a sealed interior of the user interface member is a preferred embodiment. For this purpose, the distal opening which is shown in FIG. 2 can be closed, e.g. via the user interface member part or chassis 1670 mentioned further above which will be explained further below. The electrode system 1345 may provide a large surface area of sensitivity for the capacitive sensor formed by the controller 1340 in combination with the electrode system 1345. The sensor may be or may comprise a (low-power) controller which monitors an electrical characteristic input to the controller from the sensor electrode 1345. The sensor controller 1340 is expediently configured to perform a sensor measurement or acquire a sensor signal with a frequency (polling frequency) or response rate. The frequency or response rate may be less than or equal to one of the following values: 10 Hz, 5 Hz, 4 Hz, 3 Hz, 2 Hz, 1 Hz. The power consumption of the sensor 1340 may be less than or equal to one of the following values: 15 μA, 10 μA, 8 μA, 7 μA, 6 μA, 5 μA, 4 μA, 3 μA. It will be appreciated that the power consumption depends on the frequency with which measurements are taken. The more often the sensor is polled, i.e. a measurement is conducted, the higher the power consumption will be. The order of magnitude of the power consumptions mentioned above may apply for the frequencies stated above. The frequencies are particularly suitable, if the user proximity detection unit 1330 monitors proximity to the setting surface of the user interface member. Since the time between dose setting and dose delivery is comparatively high as the user needs to switch between the two surfaces for operating the system or the device and/or since setting a dose usually takes more time as the set dose needs to be verified by the user inspecting a window and/or adjusted, smaller frequencies may be sufficient. In case the delivery surface is monitored by the sensor the frequency may have to be greater than or equal to one of the following values: 40 Hz, 50 Hz, 60 Hz, 70 Hz, 80 Hz, 90 Hz, 100 Hz. This involves a higher power consumption, e.g. greater than or equal to one of the following values: 30 μA, 40 μA, 50 μA, 60 μA, 70 μA, 80 μA, 90 μA, 100 μA. Higher frequencies are especially suitable, if the signal generated by the user proximity detection unit is used to switch the system to the state of higher power consumption, in particular to switch the motion sensing unit to the operational state, as the unit may have to be operational during the dose delivery operation which occurs pretty close to the touching of the delivery surface. If proximity to the delivery surface is monitored for another purpose, e.g. for activating another function of the system, such as related to a communication unit (see below) a smaller frequency or response rate as specified further above may be suitable. The sensor controller 1340 and the control unit 1100 may be mounted on the same surface of the carrier 3000 or on different surfaces.

A radial width or diameter of the user interface member 1600 as seen from the exterior of the member, e.g. in top view onto the delivery surface 1620, may be less than or equal to one of the following values: 2.5 cm, 2 cm, 1.5 cm. Alternatively or additionally, the radial width or diameter of the user interface member may be greater than or equal to one of the following values: 0.5 cm, 0.7 cm. The radial extension may be determined relative to the rotation axis of the user interface member during dose setting or relative to the main longitudinal axis or central axis of the user interface member, which axes may coincide. The length or axial extension of the user interface member 1600 may be less than or equal to one of the following values: 2.5 cm, 2 cm, 1.5 cm. Alternatively or additionally, the length or axial extension of the user interface member 1600 may be greater than or equal to one of the following values: 0.5 cm, 0.7 cm. For example, the radial width of the user interface member can be 18 mm and the length can be 19 mm.

The electronic system 1000 further comprises a communication unit 1400, e.g. an RF, WiFi and/or Bluetooth unit, e.g. a Bluetooth low energy (BLE) unit. The communication unit 1400 may be provided as a communication interface between the system or the drug delivery device and an external device, such as other electronic devices, e.g. mobile phones, personal computers, laptops and so on. For example, dose data may be transmitted by the communication unit to the external device and/or synchronized with the device. The dose data may be used for a dose log or dose history established in the external device. The communication unit may be provided for wireless communication. An attempt to establish communication with the external device may be included within the operation routine of the electronic system, especially after the motion sensing unit has measured a relative movement, e.g. indicative for the dose delivery operation. Also, the electronic system 1000 may comprise a functionality where the communication unit may be separately actuated, e.g. to perform a data synchronization with the external device, e.g. in case an attempt made to establish communication with the external device has failed or such a device hasn't been available. This functionality may be triggered or actuated via the delivery surface for example, especially if no touching of the setting surface preceded the signal indicating proximity of the delivery surface within a predetermined time, e.g. 10 s. The dose data may be temporarily or permanently stored within the electronic system, e.g. only the latest dose data relating to the latest delivery operation or dose history data, which contains dose data, such as date, time and or size, for one dose or a plurality of doses, e.g. all doses, which have been delivered.

As already discussed, the user proximity detection unit, e.g. the sensor controller 1340, is configured to output a signal, e.g. a proximity signal, which can be used to switch or to trigger switching of the electronic control unit 1100 or the electronic system to a state of higher power consumption. The proximity signal may be fed to or detected by the electronic control unit 1100 which then may issue commands or signals to influence the operational state of the electronic system. In the state of higher power consumption, the motion sensing unit 1200 and/or the communication unit 1400 may be operational. The proximity signal may be the signal indicative that the user is close to the setting surface (setting signal). Instead of using the proximity signal for the setting surface to wake-up the motion sensing unit and/or the communication unit, this signal can be used to increase the power consumption for the monitoring of the delivery surface, e.g. by increasing the frequency with which the sensor controller 1340 measures or polls for proximity to the delivery surface 1620. The proximity signal for the delivery surface (delivery signal) which indicates that the user is close to that surface can be used to render the motion sensing unit and/or the communication unit operational. An additional or alternative possibility is that the delivery signal causes activation of the communication unit 1400, e.g. only when a setting signal precedes the delivery signal within a predetermined time interval or, preferably only, without a preceding setting signal within a predetermined time interval. This proximity signal may be used to initiate a data synchronization event with an external device separate from a dose delivery operation. Thus, the motion sensing unit may be operational when the communication unit is rendered operational or not.

Electronic system 1000 is configured to be connected, preferably releasably, to a drug delivery device unit as an add-on unit or module. The drug delivery device unit may be electronic free. Accordingly, all electronics may be provided in the electronic system. The drug delivery device unit may be disposable. That is to say, the unit can be disposed of after a reservoir of the unit has been emptied using the drug delivery device comprising the unit and the system 1000. The electronic system 1000 can be detached from the drug delivery device unit and be reused for another drug delivery device unit. The drug delivery device unit is preferably configured as fully functional on its own, i.e. it could be operated for setting a dose to be delivered and deliver the set dose. One exemplary unit is the one depicted in FIG. 1. The electronic system may be a pure add-on to an, otherwise, fully functional unit. Alternatively, a drug delivery device may comprise the electronic system as an integral part, i.e. a part which is disposed of together with the remainder of the device and/or necessary such that the device can be operated for setting and delivering a dose of drug, e.g. because without the electronic system the drug delivery device unit would lack a surface accessible for the user for conducting a dose setting operation or a dose delivery operation.

For a connection to the drug delivery device unit, the electronic system may comprise one or more connection features 1615, e.g. snap features. The respective connection feature is arranged in a distal portion of the user interface member 1600, e.g. in the interior of the member. As noted above, the user interface member part may close the proximal end of the user interface member 1600. In this case, the connection feature 1615 are expediently provided on the user interface member part which may be rotationally and/or axially secured to the user interface member body 1605. The system 1000 is expediently configured to be mechanically connected, either permanently or removably, to a member of the drug delivery device unit such as a member of the dose setting and drive mechanism, e.g. to the drive sleeve or the dose knob and/or the injection button of the unit discussed in conjunction with FIG. 1. The system, e.g. via the user interface member body 1605, can be rotationally and axially locked to the member of the drug delivery device unit. The member to which the system is connected is expediently movable relative to the housing 10 during dose setting and dose delivery, e.g. rotationally and/or axially during setting and, e.g. only, axially during delivery. The member to which the system is connected can engage the piston rod, e.g. threadedly. The dose knob and the drive sleeve of the unit in FIG. 1 may be formed integral or act as a single member during dose setting and dose delivery. During dose setting, the drive sleeve may be selectively rotationally locked to a dial sleeve of the dose setting and drive mechanism such that the dial sleeve and the drive sleeve co-rotate during dose setting, e.g. by a clutch, and the dial sleeve rotates relative to the drive sleeve during dose delivery. The dial sleeve may be the number sleeve. The relative rotation between dial sleeve and drive sleeve during dose delivery may be measured by the motion sensing unit. However, it will be readily apparent to those skilled in the art that the disclosed concepts will also work with dose setting and drive mechanisms having different functionalities.

The electronic system 1000 as depicted in FIG. 2 comprises a connection detection unit 1700. The connection detection unit 1700 is configured to detect whether the electronic system is connected to a drug delivery device unit. The system is configured, such that, in case a connection to the drug delivery device unit is detected, the electronic system is switched to a state of higher power consumption. In case the connection detection unit detects a connection to a drug delivery device unit, a detection signal may be generated or triggered. The electronic system may be configured such that, in response to the detection signal, the electronic system 1000 is switched to the state of higher power consumption.

For example, the connection detection unit 1700 may cause the power supply 1500 to be conductively connected to other components or units of the electronic system, thereby enabling these components or units to function, when appropriately activated, e.g. by the electronic control unit. Specifically, when connection to the drug delivery device unit is detected, power from the power supply could be provided to the respective component or units. When no connection is detected, the power supply may be disconnected from the respective component or unit of the electronic system. Hence, the connection detection unit may be or may be connected to an interrupter which shuts off the power supply unless the connection to the device unit is detected.

Alternatively or additionally, the connection detection unit 1700 is configured to switch the user proximity detection unit 1330 to a state of higher power consumption, e.g. a state where the proximity or use detection unit could be operated. Before being switched to the state of higher power consumption by the connection detection unit, the proximity or use detection unit may be non-operable in a first state. The connection detection unit may trigger activation of the capacitive sensor or the associated sensor controller, e.g. for sensing proximity to the setting surface and/or the delivery surface, preferably with a low frequency.

Hence, the connection detection unit 1700 may be connected to the electronic control unit 1100 and cause the respective units to be activated or switch to the state of higher power consumption via the electronic control unit or it may be connected directly to the respective unit, e.g. the sensor controller 1340, which should be switched to the state of higher power consumption by the connection detection unit. The connection detection unit 1700 may comprise a switch 1710, such as a micro (force) switch, for example. The switch may be arranged on a side of the carrier 3000 facing towards an opening of the user interface member body 1605. The opening may be designed to receive the member of the device unit when the device unit and the system are connected. The switch 1710 may be arranged to mechanically contact or mechanically interact with a member of the drug delivery device unit and/or arranged to be triggered by the member. The member may be the same member to which the electronic system 1000 is connected via the connection feature(s) 1615. When the switch is triggered, the use detection unit or user proximity detection unit may be rendered operable. In this way, power drain of the unit during storage may be avoided or at least reduced substantially.

For example, when the system comprises a capacitive sensor as has been discussed above for the user proximity detection unit, it may be necessary to conduct measurements periodically, e.g. with a frequency as mentioned above, when the sensor operates to check whether a user is close to the relevant surface. Every measurement draws electrical power from the power supply. Thus, when the system is not connected to a drug delivery device unit, such a power drain would be completely unnecessary and the connection detection unit may switch the capacitive sensor on, e.g. to operate at a low frequency as has been discussed above. Hence, the connection detection unit improves power management, especially during terms of storage of the electronic system when being separate from a drug delivery device unit.

We note that a connection detection unit 1700 as discussed above is not only suitable for the user proximity detection unit 1330 but also for other units of an electronic system such as the electronic control unit, the motion sensing unit, the communication unit or even other units. For example, the connection detection unit may also operate with a setting sensor, a delivery sensor and/or a wake-up unit whatever is feasible in the specific situation. When the connection detection unit does not detect that the system is connected to a device unit, the power consumption may be zero or smaller than the power consumption when only the use detection unit of the electronic system is active.

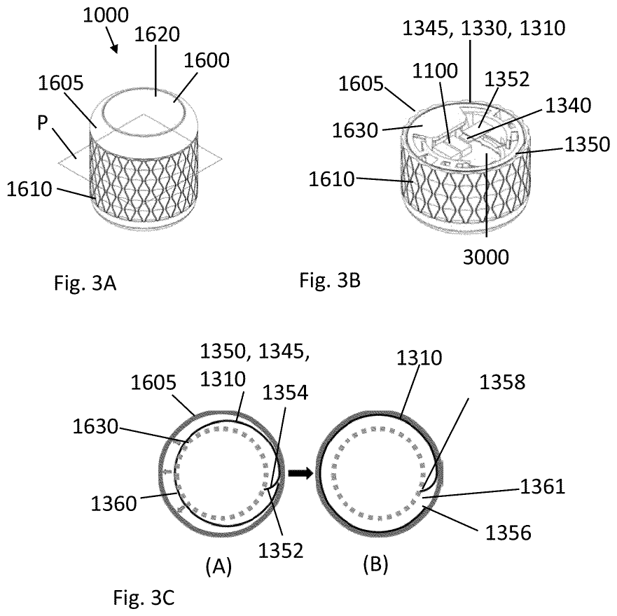

FIGS. 3A to 3C illustrate an embodiment of the electronic system 1000 and/or the electrode system 1345.

FIG. 3A illustrates a schematic perspective view of an electronic system 1000 for a drug delivery device, such as to be integrated into the device or to be connected as an add-on to a device unit, e.g. the device or device unit 1 which has been discussed further above in connection with FIG. 1. The general setting is as has been explained above already. Therefore, this description focuses on what has not been discussed already.

FIG. 3A shows the user interface member 1600 with the setting surface 1610 and the delivery surface 1620 which are formed by the user interface member body 1605, which is preferably a plastic or otherwise electrically insulating body. The setting surface 1610 is expediently structured to facilitate that the skin of the user's finger(s) can get as close as possible to the electrode system 1345 of the user proximity detection unit 1330. However, the user interface member body 1605 is preferably circumferentially closed. Particularly, the setting surface may be a continuous surface. There may be no openings in the radially facing and angularly extending setting surface. The same may hold for the axially facing and radially extending delivery surface 1620. Thus, the user interface member body 1605 is also preferably proximally closed. This facilitates provision of a sealed interior within the user interface member 1600 to house one or more electrical or electronic components as has been discussed already. Further, in FIG. 3A, a plane P is shown which visualizes the plane along which the section is taken in the sectional perspective view in FIG. 3B.

FIG. 3B illustrates the user interface member body 1605. Further, the conductor carrier 3000 is shown with the electronic control unit 1100. A part 1630 is retained in the user interface member body 1605 and, preferably fixed to the body, e.g. rotationally and/or axially. The part 1630 may be a spacer component 1510 (not shown) which is discussed later on below. The spacer component may define (mounting) space for electronic components on the conductor carrier 3000 within the user interface member and/or maintain distance between the electronic components from other components in the interior, such as from the power supply. As an alternative to the spacer component, part 1630 may be a user interface member part or chassis 1670 (not shown in this representation) which may delimit the interior of the user interface member, e.g. distally. For the present purposes, discussing a general part 1630 is sufficient. The part 1630 can have a circumferentially extending and, preferably, circumferentially closed outer surface. The user interface member 1605 has an inner surface facing the outer surface of the part 1630. The inner surface may have a cylindrical configuration. Between the outer surface of the part 1630 and the inner surface of the user interface member body 1605 the electrode system 1345 or at least a sensing region thereof, such as the setting sensing region 1310 is arranged. An electrode arrangement 1360 (more details on suitable electrode arrangements are discussed further below) is provided on a flexible conductor carrier 1350 of the electrode system. The flexible conductor carrier 1350 and the electrode system 1345 may conform to the inner surface of the user interface member body 1605, particularly the surface of the same wall defining the setting surface 1610 but opposite from the setting surface. The circumferential extension of the flexible conductor carrier 1350 when conformed to the inner surface is expediently greater than or equal to one of the following values: 270°, 355°, 360°, 400. Alternatively or additionally, the circumferential extension is less than or equal to one of the following values: 540°, 450°. Having a circumferential extension in the specified ranges, e.g. greater than 270° and less than or equal to 540° may provide an electrode arrangement over a huge proportion of the operating surface, in this case the setting surface 1610. Having an extension of greater than 360° enables that a region of the flexible conductor carrier 1350 is radially, e.g. radially outwardly, covered by another region of the flexible conductor carrier 1350 which facilitates protecting sensitive regions of the conductor carrier by another region of the conductor carrier.

The electrode system 1345, preferably, has a plurality of conductive electrode tracks running along the electrically insulating conductor carrier 1350. The flexible conductor carrier 1350 together with the electrode arrangement of the electrode tracks running thereon may form a sensor electrode system, e.g. a flexible printed circuit board. The entire electrode system 1345 may be flexible. The flexible conductor carrier 1350 has a contact connection region 1352. In the contact connection region 1352, one or more electrically conductive terminals may be provided, where each terminal may be associated with or part of one electrically conductive electrode track of the electrode system. The contact connection region 1352 may be configured to be connected to or may comprise an fcc connector. Via the contact connection region 1352, the electrode system 1345 can be conductively connected with the sensor controller 1340 which is arranged on the conductor carrier 3000, either directly or indirectly, such as via conductors on the conductor carrier 3000 or via a connector to the sensor controller 1340. This provides operative connection of the sensor controller 1340 to the sensor electrode system 1345 which comprises the electrode arrangement and the flexible conductor carrier 1350. Sensor controller and electrode system together form a capacitive sensor as noted above already. The contact connection region 1352 of the flexible conductor carrier with the associated electrode tracks may extend inwardly from the outer surface of the part 1630, e.g. in the radial direction and/or as seen in a direction perpendicular to the axis of the user interface member 1600 which may run perpendicular relative to the carrier 3000. The electrode system 1345 or the flexible conductor carrier 1350, when arranged in the user interface member 1600, expediently has a deformed configuration or shape. That is to say, the shape or the configuration has been changed from a non-deformed or first configuration or shape to a deformed or second configuration or shape for arranging the system in the user interface member. The electrode system 1345 with the flexible conductor carrier 1350 and the electrode arrangement, expediently, is elastically deformable such that the electrode system or the flexible conductor carrier, when applied in the user interface member 1600 tends to resume its non-deformed shape on account of the intrinsic elastic restoring force tending to re-establish the non-deformed configuration or shape. The elasticity may be provided by either the electrode arrangement or the flexible conductor carrier or by both. The elastic restoring force exerted by the electrode system or the flexible conductor carrier 1350 may be used to conform the electrode system or a region thereof to an internal surface or a wall of the user interface member body 160. In the present case, the inner surface of the user interface member body 1605 which faces away from the setting surface 1610, particularly in the radial and/or inward direction, defines the surface to which the electrode system of the flexible conductor carrier conforms. The non-deformed configuration of the flexible conductor carrier may be a flat or plane configuration. That is to say in the non-deformed configuration the flexible conductor carrier may be oriented along a plane.

FIG. 3C illustrates the influence or use of the elastic restoring force using two different representations, i.e. A and B. Representation A shows a state when the sensor electrode system 1345 has been introduced into the user interface member body, e.g. through the distal opening. In order to introduce the sensor electrode system into the user interface member body, the sensor electrode system has been deformed from the first configuration, e.g. where it has an extension greater than the width of the opening in the user interface member body to the second configuration, e.g. where the extension is less than the width. The first configuration may be the non-deformed configuration. The second configuration may be a deformed configuration and/or an intermediate configuration during assembly of the electronic system. In the second configuration the electrode system 1345 expediently conforms, at least in one or more regions to an outer surface of the part 1630. Thus, the outer diameter of the part 1630 may determine a dimension of the electrode system, e.g. its diameter, in the second configuration. The contact connection region 1352 extends inwards, e.g. radially inwards, relative to and/or originating from the sensing region 1310. The contact connection region 1352 is expediently conductively connected to the sensor controller 1340 as described previously, which is not shown here in detail.

The situation depicted in FIG. 3C, representation A, may be a situation which occurs during the assembling of the electronic system. A subassembly comprising the electrode system and the part 1630 may be introduced into the user interface member body 1605, e.g. such that the subassembly is completely received in the user interface member body 1605. While being on the exterior of the user interface member body 1605, the deformed electrode system 1345 may be maintained in contact with the outer surface of the part 1630, e.g. by a tool or manually, preferably also during the introduction process. After the subassembly has been introduced into the interior of the user interface member body 1605, the electrode system may be allowed to relax, e.g. by removing the tool, towards its non-deformed configuration and engage the inner wall of the user interface member body 1605, e.g. the surface which is opposite of the setting surface 1610. The situation shown in representation B may be a situation when the electronic system transitions by way of the intrinsic elastic restoring force from the intermediate configuration towards a final or third configuration it has in the assembled electronic system 1000. Clearly, in representation B, the outer surface of the electrode system 1345 conforms to the inner surface of the user interface member body 1605, particularly along the extension of the sensing region 1310, e.g. at least for 340° or more, e.g. for at least: 345°, 350°, 355°.

The electrode system 1345 has two opposite ends, a first end 1356 and a second end 1358, e.g. angular ends, especially when positioned within the user interface member body. In the second or deformed configuration, when the electrode system has been introduced into the user interface member body 1605 and/or in the intermediate configuration, there may be a region 1354 in which the electrode system overlaps angularly with itself. That is to say there is an angular position, where when proceeding from the interior of the part 1630 towards the electrode system e.g. the flexible conductor carrier or the sensing region 1310, in the radial direction (e.g. outwardly), an inner surface of the electrode system is followed by an outer surface of the electronic system which, again, is followed by an inner surface of the electronic system which is followed by an outer surface of the electronic system, where the outer surface, e.g. directly, faces towards the inner surface of the user interface member body 1605. In other words, the length of the electrode system may be greater than the circumference of the part 1630. In the second configuration the electrode system or a sensing region thereof may be wound or wrapped around the part 1630 to an extent exceeding one winding, but preferably less than two windings. In the third or final configuration of the electronic system, there may be an angular clearance 1361 between two angular ends of the electronic system or the sensing region 1310 as depicted in representation B. Alternatively, in the third or final configuration, there may still be an angular overlap of the electrode system, such that the region 1354 is present as well in the final configuration (not shown) and not only in the intermediate configuration. When transitioning from the deformed configuration towards the non-deformed configuration until the still deformed or biased final configuration is reached (as the further movement towards the non-deformed configuration is blocked by the inner wall of the user interface member body 1605), the electronic system, in particular the sensing region 1310, may expand towards the inner wall until it hits the inner wall of the user interface member body as hinted by the arrows in representation A.

In this embodiment, a flat flexible electrode system may be wrapped around the part 1630. Subsequently, the body 1605 may be assembled, constraining the electrode system. The natural stiffness or resiliency of the conductor carrier 1350 will act to unwrap the electrode system, effectively biasing it outwards onto the inside of the body 1605. In this way the electrode system is able to reliably conform to a range of outer casing part sizes, which may be expected from an injection molding process due to manufacturing tolerances, for example.

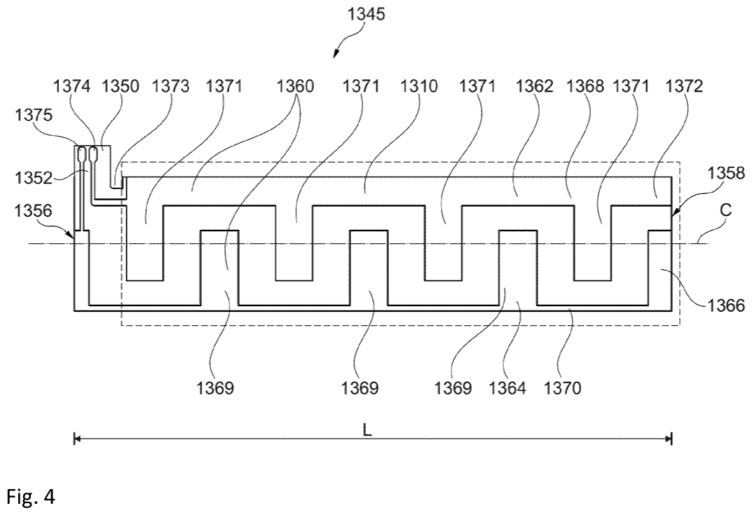

FIG. 4 shows an exemplary embodiment of an electrode system 1345 for the electronic system 1000, e.g. the one discussed above. Hence, the following discussion focuses on what has not yet been discussed, where the features discussed above do also apply to this embodiment. The electrode system 1345 is shown in its first or non-deformed configuration, e.g. the flat configuration. The flexible conductor carrier 1350 is shown with the electrode arrangement 1360 extending along the flexible conductor carrier. The two (opposite) ends 1356 and 1358 of the flexible conductor carrier 1350 or the electrode system 1345 are shown which delimit the electronic system, e.g. along the main direction of extension or length direction. The distance between the two opposite ends 1356 and 1358 may define the length L of the electrode system 1345. The length L is expediently chosen such that at least 350° of the circumferential extension of the inner surface of the user interface member body 1605 can be covered by the electrode system when being compliant to or abutting with the inner surface. As already noted, the length may even be greater than the circumferential extension of the inner surface.

The electrode system 1345 and particularly its electrode arrangement 1360 further comprises a plurality of electrically conductive electrode tracks. The depicted embodiment shows two electrode tracks, a first electrode track 1362 and a second electrode track 1364. The electrode tracks may be of metal, e.g. of copper. The electrode tracks 1362 and 1364 extend along the flexible conductor carrier 1350. The electrode system 1345 has a sensing region, in this case a setting sensing region 1310. Both electrode tracks 1362 and 1364 extend within the sensing region 1310. In the depicted configuration, the width (i.e. the extension perpendicular to the length or the main longitudinal direction of the sensing region 1310) of the sensing region 1310 is constant along the main longitudinal direction. The sensing region 1310 has a rectangular shape in the depicted embodiment and is highlighted by the dashed rectangle in FIG. 4. One of the first and the second electrode tracks, e.g. the second track 1364 or the first track 1362, in the sensing region 1310 defines or forms a sensing electrode 1366 (in the depicted embodiment electrode track 1364 forms the sensing electrode). The other one of the first and the second electrode tracks (in the depicted embodiment electrode track 1362), in the sensing region, defines or forms a reference electrode 1368. We note that which electrode track, 1362 or 1364, provides the sensing electrode and which provides the reference electrode may also be reversed.

During operation, the electrodes 1366 and 1368 are configured to be provided with different electrical potentials, e.g. positive potential for the sensing electrode and negative potential for the reference electrode or vice versa (a potential difference with potentials of the same sign may be sufficient though), such that an electrical field forms due to the different potentials between the two electrodes, sensing electrode and reference electrode. Variations in that electrical field and/or variations in the capacitance between the electrodes may be evaluated by the sensor controller 1340, with which the sensing electrode and/or the reference electrode is connected conductively.

The sensing electrode 1366 comprises a plurality of sensing electrode portions 1369. These portions are disposed sequentially along the sensing region 1310 as seen in the length or main direction and spaced from one another. The portions 1369 are oriented in a direction which is perpendicular to the main direction of extension of the sensing region and/or oriented along the axial extension of the setting surface 1610 of the user interface member body 1605 when the electrode system is arranged in the body. The portions of 1369 are oriented parallel to one another. The portions 1369 may have identical configurations. The last portion 1369 close to the end 1358 also may have a configuration as wide as the other ones and/or cooperate with the portion of the electrode track 1364 close to the end 1356 to form a combined sensing electrode portion of the same or a similar width as the other continuous electrode portions 1369 once the electrode system 1345 has been positioned in the user interface member body 1605. The portions 1369 are conductively connected by a connecting portion 1370 of the electrode track 1364. The portions of 1369 protrude from the connecting portion 1370. Thus, in the depicted embodiment, the sensing electrode 1366 may have a comb-like configuration.

The reference electrode 1368 comprises a plurality of reference electrode portions 1371. These portions are disposed sequentially along the sensing region 1310 and spaced from one another as seen in the length or main direction. The portions 1371 are oriented in a direction which is perpendicular to the main direction of extension of the sensing region and/or oriented along the axial extension of the setting surface 1610 of the user interface member body 1605 when the electrode system is arranged in the body. The portions of 1371 are oriented parallel to one another. The portions 1371 may have identical configurations. The portions 1371 are conductively connected by a connecting portion 1372. The portions 1371 protrude from the connecting portion 1372. Thus, in the depicted embodiment, the reference electrode 1368 may have a comb-like configuration.

The reference electrode 1368 and the sensing electrode 1366 are arranged such that they are interleaved with each other. Particularly, the electrode portions 1369 and 1371 may be alternatingly disposed along the main longitudinal direction of the sensing region 1310. That is to say, when traveling along the main direction of extension of the sensing region, preferably along the line C (e.g. the centerline of sensing region 1310), sensing electrode portions 1369 and reference electrode portions 1371 may alternate. Specifically a sensing electrode portion is followed by reference electrode portions which, again, is followed by a sensing electrode portion and so on. The sensing electrode 1366 and the reference electrode 1368 may be configured such that in the sensing region 1310 the distance between the sensing electrode and the reference electrode—the distance may be measured to the closest portion of the respective other electrode—is expediently constant or does not vary significantly. However, other configurations are also possible, e.g. with varying distances between the sensing electrode and the reference electrode within the sensing region 1310. The distance between the reference electrode and the sensing electrode in the sensing region should ideally be chosen such that the target (e.g. the finger(s) of the user) can be reliably detected.

The reference electrode portions and the sensing electrode portions are preferably configured alike. For example they may have the same length (the distance by which they protrude from the respective connecting portion) and/or width (the dimension perpendicular to the extension away from the respective connecting portion). The ends of reference electrode portions and sensing electrode portions may face or be oriented in different directions. Connecting portions 1370 and 1372 are oriented along one another. The sensing electrode portions may be proximally oriented or distally oriented. The reference electrode portions may be oriented in the opposite direction, e.g. distally or proximally. In FIG. 4, the upward direction may be the proximal direction (into which sensing electrode portions 1369 are oriented). The connecting portions may run parallel to one another. The reference electrode portions may be evenly distributed along the sensing region. The sensing electrode portions may be evenly distributed along the sensing region.

The contact connection region 1352 of the flexible conductor carrier 1350 in the depicted embodiment is a flexible and/or foldable tab. The tab may be flexible so as to be deflected or folded relative to the sensing region 1310. Between the contact connection region and the sensing region, a cutout 1373, e.g. a notch, may be provided in the flexible conductor carrier. This facilitates easier bending or flexing of the tab relative to the remainder of the conductor carrier 1350. In the depicted embodiment, the contact connection region 1352 is close to the end 1356 of the flexible conductor carrier 1350. Specifically, the contact connection region, e.g. the tab, may define a portion of the end of the flexible conductor carrier. It should be noted, however, that other configurations of the contact connection portion 1352 could also be used, such as the contact connection portion being within the sensing region 1310 or remote from both ends or in different configurations. In the contact connection region 1352, the width of the electrode tracks is narrower as compared to the electrode portions and/or the connecting portions in the sensing region. For example, the respective electrodes track 1362 and 1364 may be connected to terminals 1374 and 1375 of the first electrodes track 1362 and the second electrodes track 1364 via intermediate portions which are narrower than the portions of the associated electrodes in the sensing region, e.g. the electrode portions of the sensing electrode 1364 and the reference electrode 1368. The terminals 1375 and 1374 may be configured to be coupled to a plug-in connector, such as an fcc plug-in connector or a slimstack connector. Via the terminals 1375 and 1374, electrode tracks 1362 and 1364 may be conductively connected to different terminals or channels of the sensor controller 1340.

In order to achieve controlled sensitivity for the capacitive sensor, it is advantageous for the sensor controller to have a clear ground plane relative to the sensing electrode 1366. The ground plane may be provided by the reference electrode 1368. Having the reference electrode close to the surface which should be monitored is advantageous in terms of a more homogenous sensitivity of the sensor as opposed to a reference or ground potential further in the interior of the system. For example, during operation, an electrical field, e.g. a static or dynamic field, may be applied to the sensing electrode and the reference electrode. When the field changes, the capacitance of the sensor changes. The more homogeneous the field, the more homogenous will be the sensitivity of the sensor. The depicted arrangement has proven to be particularly suitable in terms of a homogenous sensitivity. For a cylindrical arrangement (as in the user interface member body 1605) it was discovered, that a particularly good or homogeneous sensitivity can be achieved with interleaving electrode tracks 1362 and 1364 as depicted in FIG. 4, where the reference electrode and the sensing electrode interleave. The exact dimensions and quantity of the tracks or electrode portions in the sensing region may be varied, e.g. to adapt to the detection of smaller hands. The sensitivity of the sensor with the shown electrode system is expediently homogeneous in the circumferential direction when monitoring proximity to the setting surface. The design depicted in FIG. 4 is particularly suitable for achieving a homogenous sensitivity for a user interface member body with a surface structure on the exterior surface.

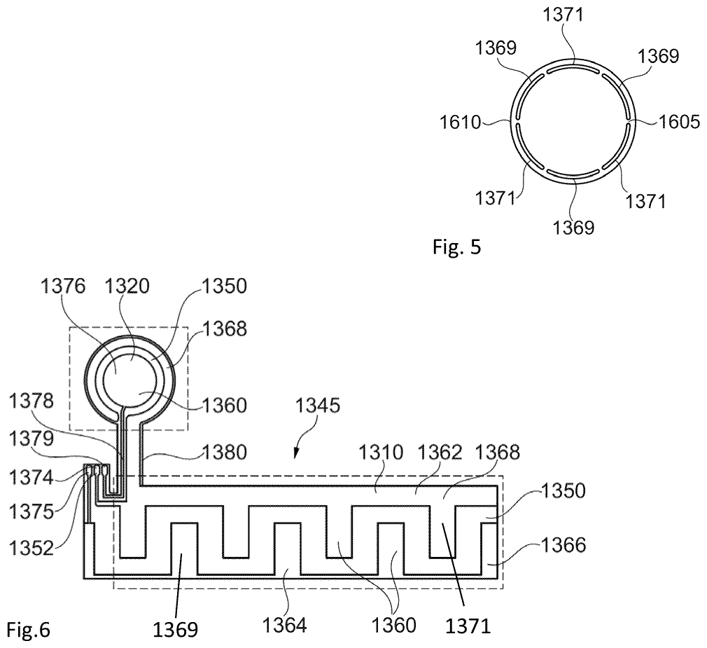

The number of reference electrode portions preferably equals the number of sensing electrode portions. The number of the reference electrode portions and/or the sensing electrode portions may be even or odd, e.g. 3, 4, 5, 6, 7 or 8. Having an odd number of reference and sensing electrode portions facilitates that, in a general cylindrical setting, e.g. when the electrode system 1345 is arranged in the user interface member body 1605, one reference electrode portion is arranged opposite one sensing electrode portion, e.g. offset by 180° in the angular direction. Having the sensing electrode portions evenly distributed along the flexible conductor carrier and the reference electrode portions evenly distributed along the flexible conductor carrier 1350 facilitates such an arrangement. When the user attempts to set a dose, it is expected that the user touches the setting surface 1610 of the user interface member at diametrically opposite locations when using the most natural combination of fingers for dose setting, i.e. index finger and thumb. The variations in the electrical field or the capacitance are expected to be particularly pronounced if electrode portions of different electrical potential—sensing electrode and reference electrode portions—are offset by 180° which is facilitated by having an odd number of sensing electrode portions and reference electrode portions. FIG. 5 illustrates this by way of example using three portions where each reference electrode portion 1369 is diametrically opposite from one sensing electrode portion 1371.

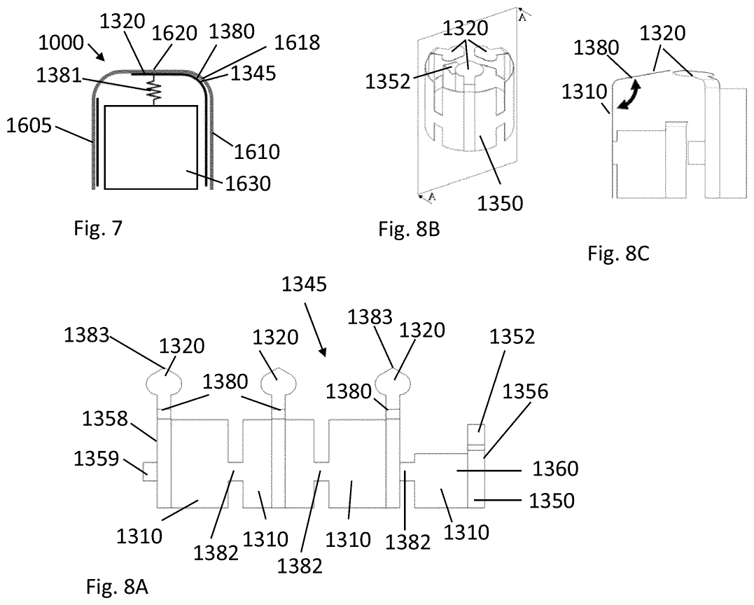

FIG. 6 shows another embodiment of an electrode system 1345, which is very similar to the electrode system 1345 discussed in conjunction with FIG. 4. Hence, the following description will focus on the differences. One difference is that the electronic system 1345 comprises two different sensing regions. Specifically, the electrode system comprises a delivery sensing region 1320, which is highlighted by the dashed rectangle in FIG. 6 in addition to the setting sensing region 1310. In the delivery sensing region 1320 a sensing electrode 1376 is arranged. The sensing electrode 1376 may be a delivery sensing electrode. A reference electrode 1368 is associated with the sensing electrode 1376 within the sensing region 1320 but electrically separated or insulated from the sensing electrode 1376, especially along the flexible conductor carrier 1350. The reference electrode 1368 and the sensing electrode 1376 are expediently arranged such that the reference electrode 1368 extends along at least the majority of the sensing electrode 1376, e.g. at least 280° of the outer circumference, within the sensing region 1320. The sensing electrode 1376 in the depicted embodiment has a circular circumference or outer contour. Other shapes may be possible as well. The sensing electrode 1376 or the sensing region may, instead of the continuous configuration without openings, also have a ring-like configuration. The flexible conductor carrier 1350 may have a hole, e.g. in the middle of the sensing electrode 1376 (not explicitly shown), particularly if the sensing electrode 1376 has a ring-like configuration. The sensing region 1320 may have a curved and/or largely circular circumference as depicted.

The sensing electrode 1376 is formed or defined by an electrode track 1378 of the electrode arrangement 1360 which extends in the sensing region 1320. Electrode track 1378 is expediently present in addition to electrode track 1362 and electrode track 1364 in the electrode arrangement 1360. The electrode tracks of the arrangement are expediently electrically insulated from one another along the entire flexible conductor carrier 1350. The reference electrode 1368 for the sensing electrode 1366 and for the sensing electrode 1376 may be formed by the same electrode track in different sensing regions. Thus, the electrode track 1364 for the reference electrode 1368 may extend in different sensing regions. The electrode tracks for the sensing electrodes 1366 and 1376 expediently extend only in one sensing region, i.e. the setting sensing region 1310 or the delivery sensing region 1320. Alternatively or additionally, the electrode track 1362 for the reference electrode 1368 is arranged between the electrode track 1378 for the sensing electrode 1376 and the electrode track 1364 for the sensing electrode 1366, preferably continuously (as depicted in FIG. 6). In the contact connection region 1352, a terminal 1379 for the contact connection to the electrode track 1378 is formed. Via terminal 1379, the electrode track 1378 or the sensing electrode 1320 can be conductively connected to the sensor controller 1340, e.g. a separate channel or input thereof, especially separate from the one for the sensing electrode 1366 and/or the reference electrode 1368.

The sensing region 1320 is connected to the remainder of the flexible conductor carrier 1350 by a connection region 1380 of the conductor carrier 1350. The connection region may extend from the remainder of the flexible conductor carrier 1350 toward the sensing region and mechanically connect the sensing region 1320 to the remainder of the flexible conductor carrier 1350. The connection region 1380 may have a width which is smaller than its length (where the length is the extension from the remainder of the conductor carrier 1350 to the sensing region 1320). The width of the connection region may be smaller than a width of the sensing region 1320. The connection region 1380 together with the sensing region 1320 may form a foldable tab of the electrode system 1345.

Having a separate electrode track 1378 for the sensing electrode 1376 (delivery sensing electrode), in addition to the reference electrode 1368 and/or the sensing electrode 1366 (setting sensing electrode) improves the delivery surface proximity sensing on account of the reference electrode and/or enables distinction between proximity to the delivery surface 1620 and the setting surface 1610 of the user interface member due to the two electrically separated electrode tracks assigned to these surfaces.

In the connection region 1380, the flexible conductor carrier 1350 may be particularly easily deformed, preferably elastically, such that the sensing regions 1310 and 1320 can be oriented into different directions, e.g. directions perpendicular to one another. The delivery sensing region 1320 can conform to an inner surface of the user interface member body 1605 which is opposite of or faces away from the delivery surface 1620. The setting sensing region 1310 may conform to an inner surface of the user interface member body 1605 which is opposite of or faces away from the setting surface 1610. The connection region 1380 can be elastically deformable such that it may bias the sensing region 1320 towards the inner surface of the user interface member body 1605 opposite the delivery surface 1620, when the electrode system is applied in the user interface member. Alternatively or additionally, a biasing member may be provided in the electrode system which biases the sensing region 1320 of the flexible conductor carrier 1350 towards the delivery surface 1620, e.g. proximally.

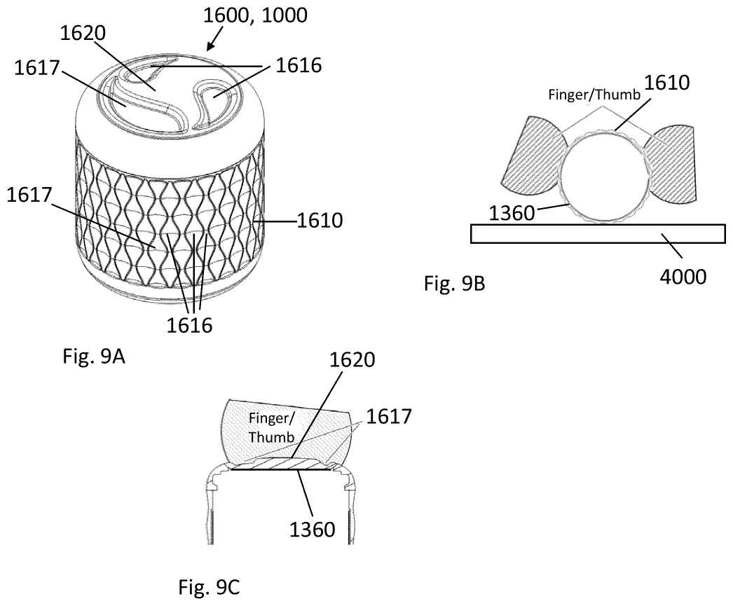

FIG. 7 schematically illustrates a biasing member 1381, e.g. a spring, such as a compression spring. The biasing member 1381 is provided to bias the sensing region 1320 towards the inner or interior surface of the user interface member body 1605 and the delivery surface 1620. The biasing member 1381 may be formed by metal component, e.g. a metal pressing. The biasing member 1381 does not have to be a helical compression spring 1381 as schematically shown depicted. Other resilient structures may be suitable for this purpose as will become apparent from the discussion further below (see the arm-like biasing portions 1540 of biasing member 1520 which may simultaneously act as power supply electrode or the electrode/biasing member 1390). The biasing member may be grounded or its force may be reacted by a part or component of the electronic system 1000 which is axially and preferably rotationally secured relative to the user interface member body 1605 or by the body itself. The part reacting the force may be the part 1630 which has been discussed above already.

It is advantageous if the electrode system 1345 is adjusted to the user interface member body such that, when the electrode system is arranged in the electronic system, the connection region 1380 conforms to an inner surface of a transition region 1618 between the delivery surface and the setting surface. The transition region 1618 may be curved on the exterior, in order to connect the radially oriented delivery surface with the axially oriented setting surface.

For the user, acting on the transition region is less likely than on the delivery surface and the setting surface for manipulations of the user interface member 1600. Hence, a sensing dedicated region is not necessarily required in the transition region of the user interface member body 1605.

It is noted that having an electrode system 1345 with just one sensing region with the respective sensing region of the electrode system being a delivery sensing region or only delivery sensing regions associated with the delivery surface is also within the scope of the present disclosure.

Providing a wrapped electrode system, e.g. a flexible printed circuit board, such as is described in conjunction with FIGS. 3A to 4 will provide sensitivity to touches on the setting surface, e.g. a cylindrical “grip” face. The addition of a foldable tab for the delivery sensing region may extend this sensitivity to the delivery surface or top face as well. An additional electrode track provides distinction between the two touch sites (setting surface and delivery surface). Crosstalk between the two channels or tracks is minimized in several ways. Firstly, the proximity between the two electrode tracks for the sensing regions is minimized wherever possible. Secondly, the ground track or reference electrode track 1362, common to both channels, is always present between the two sensing electrodes. Finally, the electrode system or the sensing region 1310 can extend or wrap further than 360° along the interior surface of the user interface member body and/or the exterior surface of the part 1630, e.g. covering up any area where the delivery sensing electrode and/or the contact connection region 1352 may be vulnerable.

The electrode system 1345 is not restricted to having one or more sensing electrodes and/or associated reference electrode(s). Rather, other electrodes, e.g. electrodes for antennas or other structures with conducting features or tracks may be implemented on the flexible conductor carrier 1350. Hence, the electrode system may comprise an electrode track for antenna, e.g. for the communication unit 1400, e.g. a Bluetooth communication unit, preferably a Bluetooth low energy communication unit (BLE: Bluetooth Low Energy).

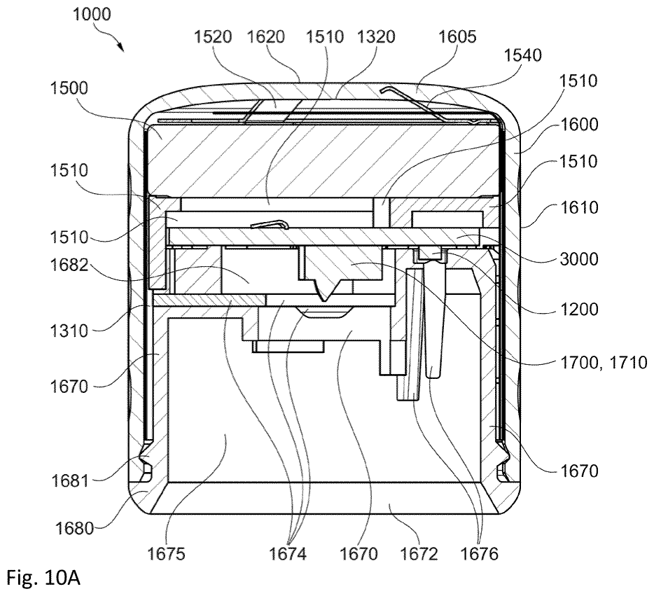

FIGS. 8A to 8C illustrates schematically another embodiment of an electrode system 1345 for the electronic system 1000. In general, system 1345 is similar to the systems described further above in connection with FIGS. 4 and 6. Hence, this description focuses on the differences.

As the previously discussed embodiments in FIGS. 4 and 6, the electrode system 1345 is shown in the first configuration or flat configuration, i.e. when it has not yet been introduced into the user interface member body 1605 and deformed. The electrode arrangement 1360 is not expressly shown in this embodiment. In general, it is envisaged that electrode tracks are provided in the same manner as in the FIG. 6 embodiment. Hence, three electrode tracks are provided, where one track is for the sensing electrode 1366, one track is for the sensing electrode 1376 and one track is for the reference electrode 1368. The specific track configuration is not shown, as this has already been discussed above. In the respective sensing region, 1310 or 1320, the associated sensing electrode 1366 or 1376 is disposed, preferably together with the associated reference electrode 1368, which again may be formed by a common track or electrode for the sensing regions 1310 and 1320.

In the depicted embodiment, the sensing region 1320 and/or the sensing region 1310 may be distributed over a plurality of separate sensing regions of the flexible conductor carrier 1350. As depicted in FIG. 8A, a plurality of separate regions, e.g. three regions, for delivery sensing regions 1320 is provided. In other words, the conductor carrier 1350 has a plurality of delivery sensing regions 1320. Each of these delivery sensing regions is connected to the remainder of the conductor carrier 1350 via an associated connection region 1380, where, preferably, one distinct connection region 1380 is associated with each sensing region 1320. The connection regions 1380 are oriented obliquely or perpendicularly to the main direction of extension of the flexible conductor carrier 1350, particularly in the first configuration. Expediently, different delivery sensing regions 1320 are configured alike. Electrode tracks 1378 and 1362 may define the electrodes in the delivery sensing regions.

Alternatively or additionally to the plurality of delivery sensing regions 1320, a plurality of, e.g. four, separate regions for the setting sensing region 1310 is provided along the main direction of extension of the flexible conductor carrier 1350. In other words, the flexible conductor carrier has a plurality of setting sensing regions 1310. Each connection region 1380 emerges from one associated setting sensing region 1310. The setting sensing regions 1310 are consecutively disposed, e.g. in a linear manner, along the main direction of extension or the length direction of the flexible conductor carrier 1350 or the electrode system 1345. The setting sensing regions 1310 are preferably of equal length, that is to say their extension along the main direction of extension may be equal. The width may also be equal, at least for a plurality of regions 1310. One region may have a different width, e.g. the one from which the contact connection region 1352 emerges, which facilitates contact connection to the sensor controller at a site (distally) offset from but radially overlapping with the delivery sensing region 1320 or the delivery surface 1620. The respective connection regions 1380 may emerge from the sensing region 1310 at different locations in different sensing regions 1310. For example, the rightmost sensing region 1320 with emerging connection region 1380 or the one closest to the contact connection region 1352, has the connection region 1380 at the beginning of the sensing region 1310 (as seen from the contact connection region 1352), the connection region 1380 for the subsequent sensing region 1310 is arranged in the middle of the sensing region 1310 and the connection region 1380 for the last sensing region or the one furthest away from the contact connection region 1352 is arranged at the end of the sensing region 1310 (as seen in the direction away from the contact connection region along the length direction). Different connection regions 1380 are expediently connected to different setting sensing regions 1310. The respective connection region 1380 is preferably foldable to allow a repositioning of the respective delivery sensing region 1320, e.g. relative to the setting sensing region(s). The respective connection region 1380, e.g. when folded, may exhibit resiliency or elastic deformability. The respective connection region 1380 may be strip-like.

The setting sensing regions 1310 are interconnected by connection regions 1382. The connection regions 1382 are oriented along the main direction of extension of the flexible conductor carrier 1350. Two adjacent setting sensing regions are connected by one connection region 1382. The connection regions 1382 may reduce the stiffness of the electrode system against bending and, hence may facilitate conforming the electrode system, particularly the sensing regions, to a circumferentially extending, e.g. cylindrical, surface. However, e.g. in case a higher elastic restoring force is required to bias the sensing region towards the setting surface, the connection regions 1382 can also be dispensed with. The connection regions 1382 are expediently smaller in width than the sensing regions 1310. The width of the flexible conductor carrier 1350, i.e. the extension perpendicular to the main longitudinal direction, may be smaller in the connection region 1382 than in the sensing regions 1310 which are connected by the connection region 1382. All electrode tracks of the electrode arrangement 1360, e.g. three electrode tracks, may run along each of the connection regions 1382. The respective connection region 1382 is strip-like.

The notches or slits provided between two adjacent sensing regions 1310, e.g. on opposite sides of the connection region 1382 connecting the sensing regions, may be advantageous, e.g. as regards conformability to a curved surface and/or for manufacturing purposes as will be discussed further below. Specifically, they may serve to rotationally lock the electrode arrangement relative to an outer surface of the part 1630, for example, and/or receive a section of a subassembly during manufacturing.

The respective delivery sensing region 1320 has an end 1383, e.g. remote from the connection region 1380 connecting the delivery sensing region to the remainder of the conductor carrier 1350. The ends 1383 may be aligned with one another in the first or non-deformed configuration. As seen from the connection region 1380 towards the end 1383 sensing region 1320 can vary in width towards the end. At first, the width increases, i.e. the sensing region 1320 may widen, e.g. up to a maximum width. Thereafter, the sensing region 1320 may narrow towards the end 1383. An outer contour of the sensing region 1320 may have a curved portion, e.g. in a region closer to the connection region 1380. The curved portion may be followed by a straight portion. The straight portion may connect the curved portion with the end 1383. The end 1383 may be pointed.

The sensing electrodes for the sensing regions 1310 are expediently formed by a respective continuous electrode track (not shown). The same holds for the sensing electrodes for the sensing regions 1320, where this electrodes track expediently is electrically insulated from the electrode track for the sensing regions 1310. The reference electrodes in the respective sensing region may belong to a common electrode track as has been described already in conjunction with FIG. 6. This track is expediently electrically insulated from the other tracks. All tracks are expediently accessible in the contact connection region 1352 for connection to the sensor controller 1340, e.g. via terminals (not shown).

A mechanical portion 1359, e.g. for fixing or aligning the electrode system 1345 relative to the user interface member body or a part for the user interface member, may be provided in the electrode system 1345. An exemplary portion 1359 is depicted on the left below the end 1358 of the electrode arrangement 1360. Alternatively or additionally, the feature 1359 may define the end 1358 of the flexible conductor carrier 1350 or the electrode system 1345.

When the electrode system is arranged in the user interface member body 1605, the setting sensing regions 1310 are disposed circumferentially along the inner surface of the user interface member body as discussed already. The ends 1383 of the delivery sensing regions 1320 may face one another and/or be oriented or directed radially inwardly. This is illustrated in FIG. 8B which schematically shows the electrode system in a situation where it is deformed for insertion or is already inserted into the user interface member body, where the body, however, is not shown. The contact connection region 1352 is shown as well. It is folded and/or bent (radially) inward. In this configuration, it can be connected to the sensor controller 1340 (not shown) which can be arranged in the interior of the user interface member when the electronic system has been assembled or manufactured.

Having a plurality of delivery sensing regions 1320 distributes the electrode surface responsible for monitoring the delivery surface on different regions of the flexible conductor carrier and/or uses a plurality of connection regions 1380. This facilitates using the elastic force provided by the flexible conductor carrier and/or the connection regions 1380 to bias the sensing regions 1320 towards the delivery surface 1320. Thus, when folded into the configuration shown in FIG. 8B the sensing regions will provide one continuous area of sensitivity for the user proximity detection unit 1330. Each tab or sensing region 1320 may feature the same electrode track configuration, and, when folded, will act as one area of touch sensitivity. This allows for the stiffness of the carrier 1350 to be better utilized e.g. during manufacturing, with multiple bends (in the connection regions) creating a greater and/or more evenly spread force (e.g. an elastic restoring force acting on the user interface member body 1605), especially when compared to the FIG. 6 embodiment.

FIG. 8C which shows the sectional view along plane A-A in FIG. 8B illustrates the biasing force which tends to maintain the region 1320 in contact with the inner wall of the user interface member body opposite the delivery surface 1620. As can be seen, in the deformed configuration where the setting sensing region 1310 is oriented along a cylindrical enveloping surface, the delivery sensing region(s) 1320 can be pivoted or moved relative to the setting sensing region 1310 due to the flexibility and/or elasticity provided by the connection region 1380. The connection region 1380 in its width direction may form the pivot axle. When the user interface member body 1605 maintains the connection region and/or the sensing region in a deflected configuration, the associated elastic restoring force biases the sensing region towards the delivery surface.

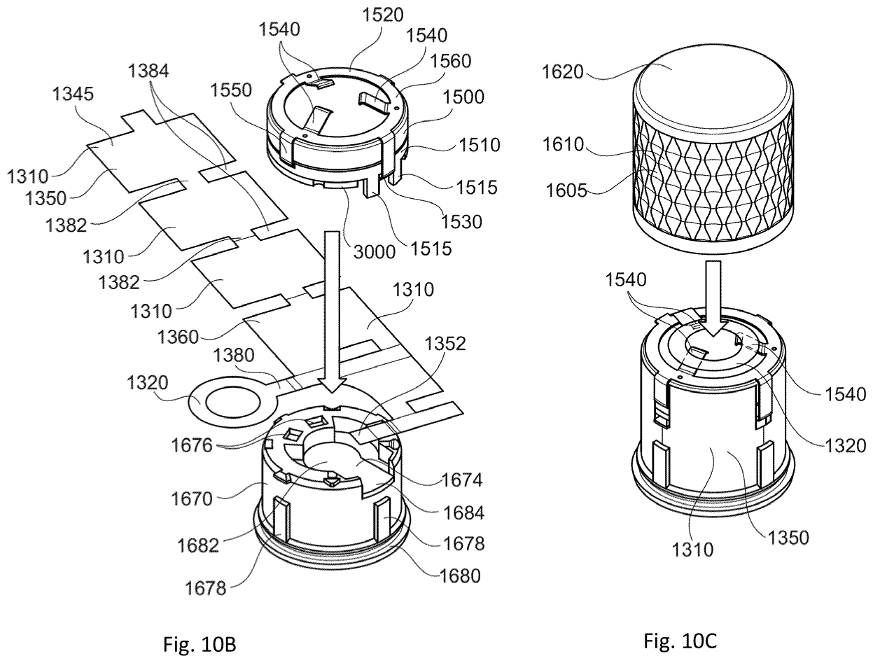

FIGS. 9A to 9C illustrate another embodiment of an electronic system 1000. FIG. 9A shows a perspective view onto the user interface member 1600 with the setting surface 1610 and the delivery surface 1620. The setting surface is provided with a surface structure 1617. Thus, the setting surface 1610 is profiled. Structure elements 1616 or recesses of the surface structure may be oriented generally axially, that is to say in the direction from the proximal end to the distal end. A plurality of consecutive structure elements 1616 or recesses may be disposed sequentially along the angular direction or circumferentially. Angular surfaces of the structure elements 1616 may have a wave-like, knurled or undulating shape. Also, the bottom surface of the respective recess may have a wave-like, knurled or undulating shape.

The delivery surface 1620 has a surface structure 1617. The structure is preferable different from the one of the setting surface. For example the structure elements 1616, e.g. recesses, may have different dimensions, such as different lengths and/or different widths. As opposed to that the structure elements 1616 of the structure of the setting surface 1610 may have equal lengths, widths, and/or equal configurations.

A surface structure 1617 on the exterior operation surface has the advantage that compliant objects like fingers will have a greater contact area with the surface than non-compliant objects like rigid plane objects, e.g. coins or metal tables. Also, compliant objects may get closer to the electrode arrangement 1360 than non-compliant objects due to the structured surface. This is illustrated by FIG. 9B which contrasts a finger or thumb which clearly has a higher contact area with the setting surface 1610 and/or is closer to the electrode arrangement 1360 than the plane object 4000. FIG. 9C illustrates the effect of the structure 1617 on the contact surface are with a compliant object for the structured delivery surface.

This embodiment features an electrode system 1345 as described in the previous embodiments. In addition, the exterior operation surface is formed with small recesses, in order to allow a compliant object (e.g. a finger) to approach the sensing electrode more closely than a rigid object (e.g. a metal table). This entails a more significant change in the electrical field or the capacitance by the compliant object than the rigid non-compliant object. On the cylindrical surface or setting surface the recesses can be provided as ridges, e.g. in any pattern, for example knurling, and also can serve to assist the user in achieving purchase or grip on the surface. On the top surface or the delivery surface, the recessed area may simultaneously act as an embossed design or branding.

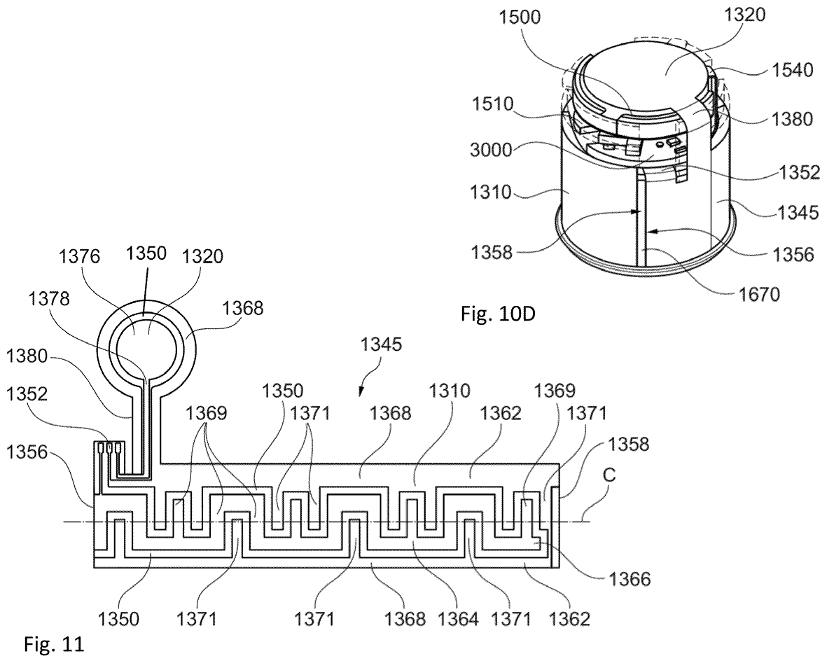

FIGS. 10A to 10D illustrate embodiments of the electronic system 1000, which in some aspects are more specific than the one discussed in conjunction with the preceding figures, and a method of assembling or manufacturing the electronic system. However, all features disclosed in conjunction with the previous figures should also apply for the system 1000 and vice versa.

FIG. 10A shows a schematic sectional view of the electronic system 1000. The system, again, comprises the user interface member 1600 with the user interface member body 1605 defining the setting surface 1610 as well as the delivery surface 1620. The conductor carrier 3000 is provided in the interior of the user interface member body 1605 and carries one or more electronic units as described above, e.g. the sensor controller 1340, the motion sensing unit 1200, the connection detection unit 1700 and/or the electronic control unit 1100. The conductor carrier 3000 is axially and rotationally secured in the user interface member, e.g. to the chassis 1670 described further below. In the sectional view of FIG. 10A, not all of the units discussed previously are explicitly illustrated but may be present nevertheless.

The arrangement of the power supply 1500 and its contact connection to electrically powered components are illustrated in more detail than in the schematic representation of FIGS. 2 and 3. The power supply 1500 is arranged between the carrier 3000 and the proximal surface of the user interface member 1600, i.e. the delivery surface 1620. A spacer component 1510 is arranged between the carrier 3000 and the power supply. Hence, the power supply 1500 is supported on the carrier 3000 via the spacer component 1510. The spacer component 1510 may have a curved interior surface in order to extend circumferentially along the (electronic) components mounted on the carrier 3000. The spacer component 1510 expediently defines a hollow between the surface of the carrier 3000 facing towards the spacer component and the power supply. In the hollow, electronic units or components on the carrier 3000 may be arranged without a risk of contacting the power supply 1500 directly. The spacer component also assists in avoiding excess mechanical loads, e.g. loads exerted by the user onto the delivery surface during the dose delivery operation, being transferred directly to the electronic units or components on the carrier 3000.

Further, FIG. 10A shows the power supply electrode 1520. The electrode 1520 is conductively connected to the power supply 1500 and to one or more electrical or electronic components, particularly via the conductor carrier 3000. The electrode 1520 has a contact portion 1530 which conductively connects the electrode to the conductor carrier 3000 (see FIG. 10B). The electrode 1520 extends from that side of the carrier 3000 which faces the power supply 1500 to that side of the carrier 3000 which faces away from the power supply 1500. On the latter side, the contact portion 1530 is arranged. However, different configurations of the contact connection between power supply and electronic component(s) can also be envisaged. The electrode 1520 has one or more biasing portions 1540 (three in the depicted embodiment). The biasing portions 1540 are elastically deflectable. The respective biasing portion 1540 can be a flexible arm, e.g. with a free end. The respective biasing portion 1540 can extend in the proximal direction oblique to the main axis of the user interface member. The respective biasing portion is provided to exert a force on the power supply 1500 such that the power supply is biased towards the carrier 3000 and/or the spacer, e.g. distally. Alternatively or additionally the biasing portion(s) is(are) provided to bias one or more sensing regions of the electrode system 1345 (see FIG. 10B, the sensing region 1310 and 1320 are illustrated in FIG. 10A only very schematically) towards the exterior operation surface of the user interface member body 1605 which should be monitored by the user proximity detection unit. The biasing portions 1540 may bias the power supply 1500 towards the carrier 3000 and the sensing region 1320 towards the delivery surface 1620. The biasing portions 1540 are connected by a ring portion 1560 and extend axially away from and inwardly relative to the ring portion 1560 (see FIG. 10B). The ring portion 1560 may be conductively connected to the power supply 1500, e.g. one terminal thereof. The electrode 1520 has a securing portion 1550. The securing portion 1550 may be designed to secure the power supply 1500 to the spacer component 1510, e.g. to form a subassembly or unit which can be handled during the assembling of the electronic system. In the depicted embodiment, this is achieved by clamping the spacer component and the power supply between regions of the electrode 1520 (not explicitly shown).

The system 1000, in addition to the user interface member body 1605, has a user interface member part or chassis 1670. References to the chassis herein should be understood as references to the user interface member part and vice versa. The chassis 1670 is, preferably rotationally and axially, locked to the user interface member body 1605, e.g. snap fitted or welded (a snap-fit with snap feature(s) 1681 is illustrated in this embodiment, e.g. close to the distal end of the user interface member). The chassis, together with the user interface member body, may define an interior of the user interface member 1600. The interior is preferably sealed, e.g. dust-tight and/or fluid-tight. For this purpose, one or more seals, e.g. o-rings, may be provided at the interface user interface member body/chassis (not explicitly illustrated). The chassis 1670 closes a distal opening of the user interface member body 1605. The chassis preferably comprises a rigid portion 1672 and/or a deformable, e.g. elastically deformable, portion 1674. The chassis 1670 may be a part formed in a 2K molding process. The connection features 1615 for connecting the system to the drug delivery device unit can be provided on the chassis, e.g. an exterior surface thereof (not explicitly shown), which may face inwardly radially.

The chassis 1670 defines a receiving space 1675 of the user interface member 1600, which space may be open in the distal direction. The receiving space 1675 is provided for receiving the member of the drug delivery device unit to which the system 1000 should be connected.

The deformable portion 1674 of the chassis, which may limit the receiving space proximally, is preferably designed to interact with a member of the drug delivery device unit and, when the unit has been connected to the system, the deformable portion 1674 is preferably elastically deformed by the member as compared to the situation when the system is not connected to the device unit. The (proximal) movement during the deformation of the deformable portion 1674 may be used to trigger the switch 1710 of the connection detection unit 1700. This will cause that the connection of the electronic system to the device unit is detected electronically and the system may be switched to a state of higher power consumption, e.g. by activating the user proximity detection unit 1330. When the system is disconnected from the device unit, the portion 1674 may resume its undeformed shape due to the elasticity, thereby causing the connection detection unit 1700 to detect a disconnection from the device unit, e.g. by the switch 1710 changing its state. As the portion 1674 is part of the chassis 1670, a direct contact between the member of the drug delivery device unit and the switch 1710 or the connection detection unit 1700 is not required which facilitates provision of a sealed interior of the user interface member 1600, e.g. for the electronics and the electrode system. The chassis 1670 preferably also comprise one or more light guide portions 1676. The light guide portion(s) may be operatively coupled to the radiation emitter and radiation sensor provided in the motion sensing unit 1200, in case radiation is used for motion sensing. Consequently, movement of a member which moves relative to the end surface of the light guide 1676 remote from the interior of the user interface member 1600, e.g. an encoder component, may cause variations in the radiation (intensity) reflected back to the radiation sensor, where this reflected radiation may be guided to the radiation sensor through the light guide portion and/or the radiation preferably was generated by the radiation emitter. In this way, the movement of the member, e.g. the dial or number sleeve, relative to the user interface member 1600 or the dose knob or button may be quantified, such as during the dose delivery operation. The light guide portions and/or the according sensors may be out of phase relative to reflective portions of the encoder component (see WO 2019/101962 A1) which has some advantages.

FIG. 10B shows the chassis 1670 and the electronic system 1345. The electrode system 1345 is very similar to the one which was described above in conjunction with FIG. 8A. We note, however, that any other electrode system, especially of the ones described herein, could be used as well. The electrode system 1345 has a plurality of sequentially disposed setting sensing regions 1310, which are connected by connection portions 1382. Not all of the portions or regions are highlighted in FIG. 10B. The electronic system 1000 also has a delivery sensing region 1320. As opposed to the FIG. 8A embodiment, only one delivery sensing region 1320 is provided similar to the embodiment depicted in FIG. 6. The electrode arrangement 1360 is not depicted in detail in this embodiment but, however, is incorporated within the design of the electronic system 1345 as the electrode tracks provide the functionality or the proximity sensing to the exterior operation surfaces(s). Slits 1384, e.g. on that side of the flexible conductor carrier 1350 which is or is to be arranged remote from the delivery sensing region 1320, e.g. as seen along the width direction, may be configured to align with or receive ridges 1678 on the outer surface of the chassis 1670, especially when the flexible conductor carrier 1350 is wrapped around the outer surface of the chassis 1670. The ridges 1678 may be axially oriented, e.g. along the proximal direction. The ridges 1678 may cooperate with the slits 1384 in order to secure the angular relative positions between the chassis 1670 and the electrode arrangement 1345 once the flexible conductor carrier 1350 or the electrode system has been wrapped around the outer surface of the chassis 1670. A radially outwardly protruding flange 1680 may be provided around the (entire) circumference of the user interface member part or chassis 1670, e.g. at the distal end. The flange 1680 may provide the distal end of the user interface member and/or a seat for the user interface member body 1605. Proximally offset from the flange that is to say towards the delivery surface 1620, the one or more snap features 1681 are provided.

In the surface of the chassis 1670 which faces towards the proximal end or towards the delivery surface 1620, a recess 1682 is provided, e.g. to accommodate switch 1710. The bottom or distal end surface of the recess may be defined by the deformable portion 1674. The sidewall may be defined by the rigid portion 1672 of the chassis.

For assembling or manufacturing the electronic system 1000, the electrode system 1345 may be provided and the carrier 3000 with electronic units or components mounted thereon. The sensor controller (not shown) may be mounted on a surface of the carrier which is provided to face towards from the chassis 1670 or on the side of the carrier which is provided to face towards the delivery surface 1620, i.e. proximally. The contact connection region 1352 can be conductively connected to the sensor controller 1340, e.g. directly or via conductors on the conductor carrier 3000. Preferably after having established the connection, the contact connection region 1352 is arranged relative to the chassis 1670 such that the region 1352 is inwardly offset relative to the outer surface of the chassis 1670. Alternatively, at first the contact connection region 1352 may be arranged as depicted in FIG. 10B and then the contact connection with the sensor control 1340 may be established. For the contact connection an fcc plug may be used. Alternatively to an fcc plug, a connector for a vertical connection, such as a slimstack connector may be used. The chassis 1670 expediently comprises a slot, e.g. radially and/or inwardly oriented, to receive the contact connection region 1352.

It is advantageous, e.g. from the manufacturing perspective, if a subassembly is provided which comprises a plurality of components or units, preferably the carrier 3000 (e.g. with the sensor controller 1340, the electronic control unit 1100, the communication unit 1400 and/or the motion sensing unit 1200), the spacer component 1510, the power supply 1500 and/or the power supply electrode 1510 as depicted in FIG. 10B. The carrier 3000 is expediently arranged to face towards the chassis and/or limit the subassembly on the side facing towards the chassis.

The contact connection to the sensor controller 1340 is expediently made when the sensor controller is part of the subassembly already. The subassembly is configured to be arranged on, e.g. placed on top of the chassis, such as onto a proximal surface thereof. The subassembly may be secured to the chassis, e.g. axially and/or rotationally. The subassembly and the chassis 1670 are configured such that the subassembly cannot be arranged on the chassis in any relative rotational orientation. Rather, in the depicted embodiment, the subassembly can be arranged on the chassis only in one predetermined relative rotational orientation. The subassembly, in the depicted embodiment the spacer component 1510 (but other implementations are possible), comprises one or more rotational locking or orientation features 1515. Features 1515 are expediently configured to cooperate with corresponding features 1684 on the chassis 1670 when the desired rotational orientation is given to allow the subassembly to be arranged on the chassis, e.g. by contacting a surface on the chassis. If the subassembly and the chassis are not in the predetermined orientation, the feature(s) 1515 may cooperate with the chassis, e.g. a surface thereof to prevent that the subassembly can be positioned onto or bear on the chassis with a bearing surface, e.g. a surface of the carrier 3000, contacting the chassis.

The corresponding feature 1684 may comprise a recess in the chassis configured to receive the feature 1515 when the chassis and the subassembly are in the predetermined rotational orientation relative to one another. The corresponding feature 1684 may be defined by a wall of the chassis 1670 delimiting a recess, e.g. recess 1682. The recess 1682 may extend from the outer surface of the chassis 1670 in the inward direction and therefore provide the corresponding feature as well as space for switch 1710. The bottom of the recess or the distal surface delimiting the recess of the corresponding feature 1684 may be formed partly or entirely by the deformable portion 1674. Alternatively or additionally to the orientation functionality, the feature(s) 1515 can rotationally lock the subassembly relative to the chassis when the subassembly has been mounted to the chassis 1670. When the chassis and the subassembly are in the correct rotational orientation, the motion sensing unit 1200 can be operatively coupled to the light guide portion(s) 1676, e.g. the motion sensing unit may face the light guide portion(s) 1676 and/or the switch 1710 can be triggered by deformation of the deformable portion 1674. The subassembly may be locked axially to the chassis 1670, e.g. by a snap fit (not explicitly shown) or be clamped in position by the user interface member body 1605 and the chassis 1670 when they are axially locked. The subassembly is expediently arranged on the chassis in the correct orientation after the contact connection region of the electrode system has been conductively connected with the sensor controller 1340 or the carrier 3000. The contact connection region 1352 may be arranged between the subassembly or the carrier 3000 and the chassis 1670.

During the assembling of the electronic system 1000, preferably after the electrical contact of electrode system 1345 and the sensor controller 1340 has been established and/or after the subassembly has been arranged on the chassis in the correct orientation, the setting sensing region(s) 1310 can be folded relative to the contact connection region 1352, e.g. such that the setting sensing region(s) is(are) oriented axially. The setting sensing region(s) may be folded downwardly or distally, e.g. so as to be oblique or perpendicular to the contact connection region 1352. The flexible conductor carrier 1350 can be wrapped around the chassis on its outer surface, such that the sensing region(s) faces in the radial direction and/or enclose the chassis circumferentially. Then, the sensing region(s) 1310 is (are) arranged along the outer surface of the chassis 1670, e.g. a surface with a cylindrical configuration. The process may continue with arranging the sensing region 1320, such that it is oriented radially and/or extends along the contact connection region 1352, the carrier 3000, the power supply 1500 and/or the proximal surface of the chassis 1670. This may be achieved by folding the connection region 1380 and/or the sensing region 1320 appropriately. When the sensing region 1320 has been moved into the desired portion, e.g. to face in the proximal direction and/or towards the delivery surface 1620, the sensing region, e.g. with a distally facing surface of that region, can bear on the biasing member(s) 1540, preferably formed by the power supply electrode 1520. This configuration is depicted in FIG. 10C.

Thus, in FIG. 10C, the sensing regions 1310 and 1320 are positioned appropriately such that they can sense proximity to the setting surface 1610 or the delivery service 1620 of the user interface member 1600. One or more portions of the power supply electrode may extend within or be angularly aligned with notches or slits in the flexible conductor carrier 1350. The slits or notches may be formed in the regions of the connection region 1382. The correct positioning of components may be visually inspected in the stage shown in FIG. 10C. The process is continued by introducing the unit comprising the flexible conductor carrier 1350, the subassembly and the chassis 1670 into the user interface member body, and fixing the chassis 1670 to the user interface member body 1605, e.g. by the discussed snap fit.

Before being introduced into the user interface member body, an elastic force which tends to reestablish the first or flat configuration of the electrode system 1345 may be reacted, e.g. by a mounting tool (not illustrated). Once the force is no longer reacted, e.g. when removing the mounting tool, the elastic restoring force may press the electrode system, particularly the sensing region(s) 1310, against the inner wall of the user interface member body 1605. This may improve the operative connection between the electrode system and the exterior operation surface. In case the elasticity of the electrode system is not given or not sufficient, the force can be provided by (another) biasing member which biases the sensing region(s) 1310 towards the setting surface 1610.

FIG. 10D illustrates a structure with the chassis 1670, the electrode system 1345 (the electrode tracks are not shown) and the carrier 3000 with the contact connection region 1352 being conductively connected to the sensor controller 1340 either via conductors on the carrier 3000 or directly. Likewise, the spacer component 1510, the power supply 1500 and power supply electrode 1540 are shown. As opposed to the representation in FIG. 10C, the delivery sensing region 1320 is a continuous region and not formed by consecutively disposed regions. However, it should be understood that the depicted features could also be employed for any other of the embodiments processed herein. In the electrode system 1345, the contact connection region 1352 and the connection region 1380 are close to one another, e.g. with an angular distance of less than one of the following values: 90°, 45°, 30°, 25°, 20°, 15°. The opposite ends 1356 and 1358 face one another in this embodiment such that small gap is formed between them, where the chassis 1670 is visible. It should be understood that, e.g. in order to protect the contact connection region 1552 and/or the connection region 1380, the flexible conductor carrier could be made longer, e.g. such that the contact connection region 1352 and/or the connection region 1380 are covered by the flexible conductor carrier. In this case, the flexible conductor carrier may wrap around more than 360° around the chassis.

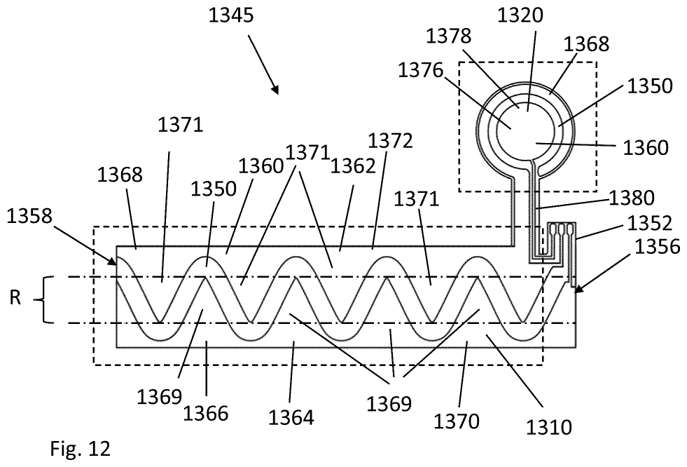

FIG. 11 illustrates another embodiment of the electrode system 1345. This embodiment is very similar to the one discussed in conjunction with FIG. 6. Thus, the following discussion focuses on the differences. The most prominent difference is the configuration of the electrode track 1362 for the reference electrode 1368. This electrode track 1362 extends on two opposite sides of the electrode track 1364 for the sensing electrode 1366 in the sensing region 1310. The electrode track 1362 extends along the electrode track 1364 in a first direction, e.g. as seen from the contact connection region 1352 and/or towards the end 1358 and in a second direction opposite and preferably parallel to the first direction. The electrode track 1362 may surround the electrode track 1364. In a transition portion, e.g. close to the end 1358, the electrode track 1362 may extend from one side of the electrode track 1364 to the other side, e.g. by passing an end of the track 1364 closest to the end 1358 of the system 1345 or the flexible conductor carrier 1350. This configuration also results in reference electrode portions 1371 pointing with their ends in different directions. Some portions 1371 will point distally and some portions will point proximally when applied in the electronic system 1000. The end of each portion preferably points towards the electrode track 1364 for the sensing electrode 1366. The electrode portions 1371 point in the same directions on the respective sides of the electrode track 1362. Sensing electrode portions 1369 and reference electrode portions 1371 are equidistantly disposed along the sensing region 1310. Electrode portions 1371 branching off the electrode track 1362 protrude into recesses defined by the electrode track 1362. This configuration of the electrode tracks facilitates having a high number of setting and reference electrode portions which alternate in the length direction of the sensing region 1310. In the connection region 1380, the electrode track 1378 for the sensing electrode 1376 in the sensing region 1320 runs between two portions of the electrode track 1364 for the reference electrode 1362.

It is noted that having an electrode track for the reference electrode on the flexible conductor carrier in addition to electrode track(s) for one or two different sensing electrodes is not necessary to implement the disclosed concepts. For example the electrode track for one sensing electrode, e.g. the one for the delivery surface, may act as reference electrode when proximity to a surface should be monitored by another sensing electrode, e.g. the one for the setting surface. Also, the reference electrode may be remote from the flexible conductor carrier in the interior. Having a dedicated electrode track in the electrode system for the reference electrode may nevertheless be advantageous. However, having a reference electrode on the flexible conductor carrier along with the sensing electrode facilitates improving or improves the sensitivity of the sensor comprising the sensor controller and the electrode system. Also, if two different sensing electrodes are provided (e.g. for setting and delivery surfaces), the reference electrode can provide a degree of isolation between the sensing electrodes, particularly if the reference electrode extends between the sensing electrodes and/or is electrically separated from both sensing electrodes along the flexible conductor carrier.

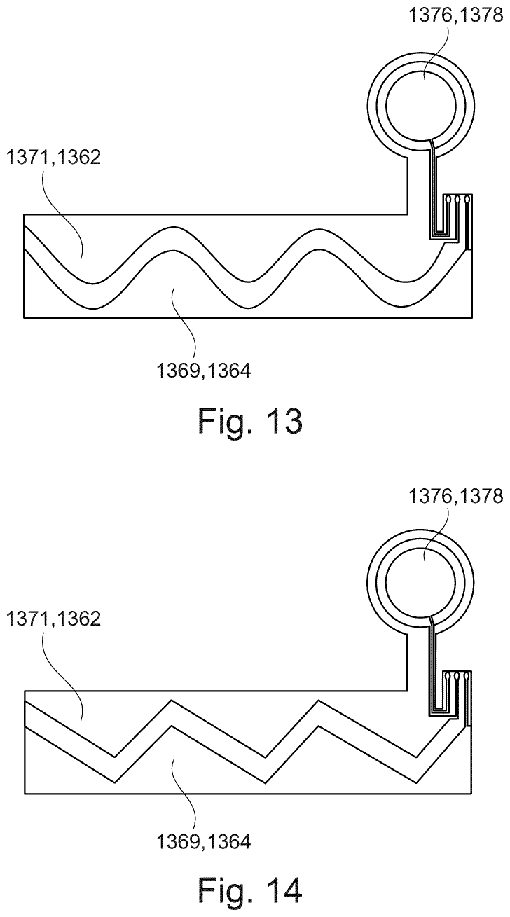

FIG. 12 illustrates an embodiment of the electrode system. This embodiment is very similar to the ones discussed in conjunction with FIGS. 6 and 11. Thus, the following discussion focuses on features which facilitate the description of the differences. However, as in the previous embodiments, features not expressly disclosed in conjunction with this embodiment may also be implemented. The electrode system 1345 has a setting sensing region 1310. The electrode system has a delivery sensing region 1320. Again, as in the previous embodiment, one of the regions may suffice, e.g. the setting or delivery sensing region. In the present embodiment, the delivery sensing region 1320 is configured in the same manner as in FIG. 6. However, a configuration as in FIG. 11 is possible as well. The delivery sensing region 1320 and the setting sensing region 1310 are connected by a connection region 1380 of the flexible conductor carrier 1350. The connection region 1380 facilitates moving the delivery sensing region 1320 relative to the setting sensing region 1310, e.g. to conform these sensing regions to two differently oriented surfaces, e.g. surfaces running perpendicularly relative to one another.

As in the previous embodiments, the setting sensing region 1310 is adapted to conform to a curved, e.g. cylindrical, surface of the user interface member body 1605, e.g. the sensing surface associated with the setting surface 1610. The delivery sensing region 1320 is adapted to conform to a plane surface of the user interface member body 1605, e.g. the sensing surface associated with the delivery surface 1620. In the setting sensing region 1310, the electrode arrangement 1360 comprises two electrode tracks, i.e. tracks 1362 and 1364. Electrode track 1362, again, defines the reference electrode 1368 and electrode track 1364 defines the sensing electrode 1366. The respective electrodes track extends along the longitudinal direction of the sensing region 1310 between the ends 1356 and 1358 of the flexible conductor carrier 1350 or the, preferably continuous, sensing region 1310. The electrode tracks 1362 and 1364 extend along one another in the sensing region, e.g. generally away from (longitudinal) end 1356 and towards (longitudinal) end 1358. The sensing electrode 1366 and the reference electrode 1368 extend along opposite ends of the sensing region, e.g. ends delimiting the region in the width direction or axial direction or perpendicularly to the longitudinal direction.

Reference electrode portions 1371 and/or sensing electrode portions 1369 of the associated electrode extend obliquely or perpendicularly to the longitudinal or length direction of the setting sensing region 1310. The reference and sensing electrode portions overlap with one another, particularly along a central and/or strip-like area R of the sensing region 1310 extending between the ends 1356 and 1358. The area R is visualized by the dash-dotted line in FIG. 12.

The respective electrode portion, that is to say sensing electrode portion 1369 and/or reference electrode portion 1371, has a (free) end. One of the electrode portions—sensing and/or reference electrode portion—may have a region overlapping with the other one of the electrode portions—reference or sensing electrode portion, respectively—and a region where the one of the electrode portions do not overlap with the other one of the electrode portions. The non-overlapping region may be closer to the connecting region connecting one of the electrode portions than the end of the other one of the electrode portions is to the same connecting region. The main extension directions of the sensing electrode portions and/or of the reference electrode portions are expediently parallel to one another. The sensing electrode portions 1369 extend away from the connecting portion 1370, which connects the sensing electrode portions conductively, and/or towards the connecting portion 1372 connecting the reference electrode portions 1371 conductively. The reference electrode portions 1371 extend away from the connecting portion 1372 connecting the reference electrode portions conductively and/or towards the connecting portion 1370. The (free) end of the respective electrode portion is remote from the connecting portion connecting the respective electrode portions. The end of an electrode portion may be a region located furthest away from the associated connecting portion, e.g. as seen perpendicular to the longitudinal direction of main extension of the sensing region or in the axial or width direction. The connecting portions 1370 and 1372 extend along opposite ends of the flexible conductor carrier 1350 or the sensing region 1310.

The (free or protruding) ends of the reference electrode portions 1371 are aligned with each other. The (free or protruding) ends of the sensing electrode portions 1369 are aligned with each other. As seen in the width direction the aligned ends may be at the same or substantially the same location. The ends of the reference electrode portions 1371 and the ends of the sensing electrode portions 1369 are offset from one another, e.g. in the width or axial direction and/or in the longitudinal direction. Thus, the reference electrode portions 1371 and the sensing electrode portions 1369 again have an interleaved arrangement as in the previous embodiments.

In the previously described embodiments, however, the width of the sensor electrode portions that is to say their dimension perpendicular to their main direction of extension, i.e. away from the connecting portion towards the (free) end, was constant along the extension of the electrode portion towards its end. Thus, the respective electrode portion had an essentially rectangular shape when seen in top view onto the portion. In the presently depicted embodiment, the reference electrode portions 1371 reduce in width as see in the direction away from the connecting portion 1372 and/or towards the end of the reference electrode portions. The same holds for the sensing electrode portions alternatively or additionally with respect to the connecting portion 1370 and the associated end of the portion. The respective electrode portion is preferably symmetrical with respect to an axis running through the free end and/or being parallel to the main extension direction of the electrode portions along the flexible conductor carrier. This axis may extend between the proximal end to the distal end of the system or the device, e.g. when the electronic system is applied in the device.

In the depicted configuration, the region of the flexible conductor carrier 1350 separating the reference electrode 1368 and the sensing electrode 1366 has a wave-like or sinusoidal shape. This is opposed to the meander-like shape in the previous embodiments. Accordingly, at a given position—e.g. at any position or in all positions but one—in a region where the reference electrode portions and the sensing electrode portions overlap, the respective electrode portions may have different extensions along the longitudinal direction. For example, the reference electrode portions may be wider than the sensing electrode portions in the region closer to the connecting portion which connects the reference electrode portions. This configuration may be beneficial for the homogeneity of the sensitivity of the sensor comprising the electrode arrangement or adjust the sensitivity of the sensor to a desired sensitivity characteristic.

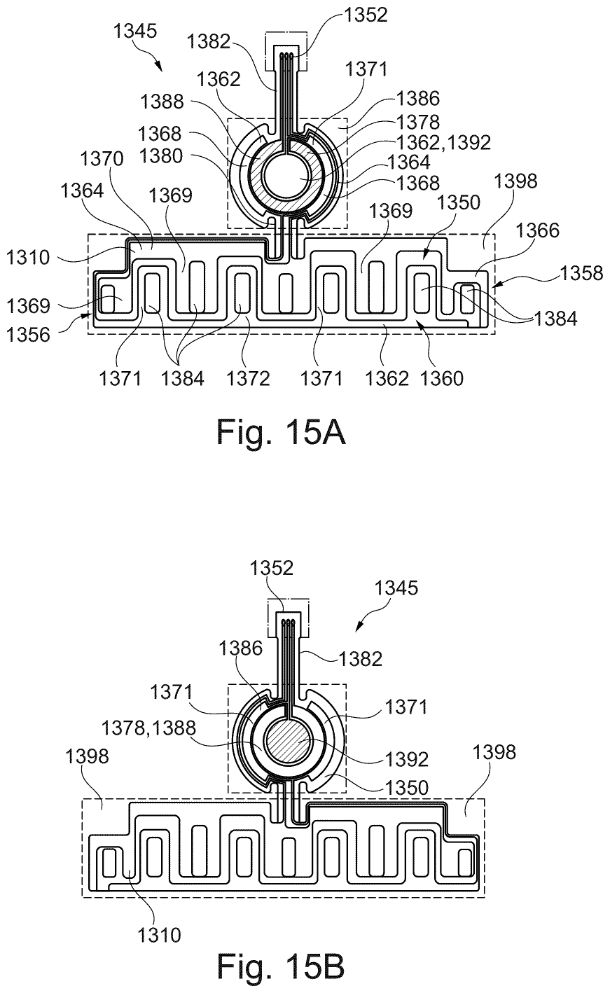

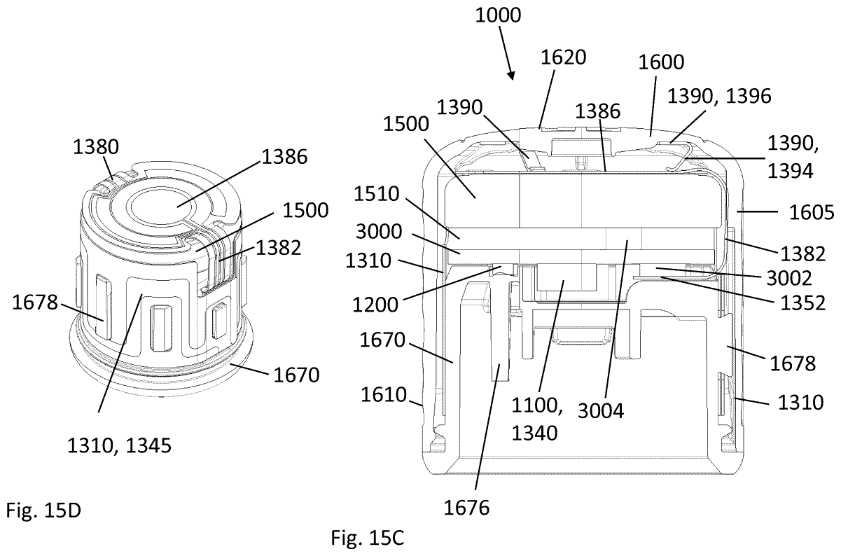

FIGS. 13 and 14 schematically illustrate further embodiments of the electrode system. The embodiments are similar to the one shown in FIG. 12. Particularly, the electrode portions may reduce in width in the direction away from the associated connecting portions. FIG. 13 illustrates a sinusoidal arrangement of the electrode portions (in particular the area separating the electrode portions 1369 and 1371 associated with different electrode tracks has a sinusoidal shape). FIG. 14 illustrates a zig-zag or sawtooth arrangement of the electrode portions (in particular the area separating the electrode portions 1369 and 1371 associated with different electrode tracks has a zig-zag or sawtooth shape).

As set forth above, the layout of the electrode tracks (for the reference electrode and the sensing electrode) may feature a range of electrode patterns or shape of the electrode portions, such as rectilinear (FIGS. 4, 6 and 11), zig-zag or sawtooth (FIG. 14) or sinusoidal repeating (FIGS. 12 and 13). The layouts of the electrode portions or the configuration of the electrode tracks is expediently chosen (e.g. by adjusting the arrangement and/or the configuration of the electrode portions appropriately) so as to have a reliable and/or improved response to a user's grip, e.g. by increasing the likelihood that the user's finger or thumb will cover both tracks or portions (reference electrode and the (respective) sensing electrode) when gripping the user interface member.

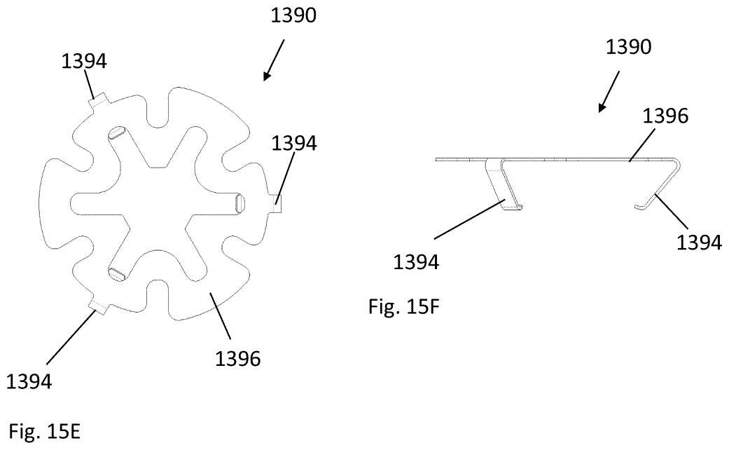

FIGS. 15A to 15F illustrate an embodiment of an electrode system, an embodiment of an electronic system using the electrode system and an embodiment of a method. Features described in the context with this embodiment may also be applied for other embodiments described herein and vice versa.

FIGS. 15A and 15B show the electrode system 1345 from opposite sides. FIG. 15B has been rotated by 180° relative to FIG. 15A. In general, electrode system 1345 is similar to the systems described further above, e.g. in connection with FIGS. 4, 6, 11 and 12. Hence, this description focuses on the differences.

The electrode system 1345 comprises the setting sensing region 1310. The setting sensing region extends between the two ends 1356 and 1358 of the electrode system. The ends 1356 and 1358 may be two opposite or remote ends of a strip-like region of the flexible conductor carrier 1350. In the setting sensing region 1310, the (setting) sensing electrode 1366 is provided by the electrode track 1364 of the electrode arrangement 1360. The sensing electrode 1366 has a connecting portion 1370. A plurality of distinct sensing electrode portions 1369 extends away from the connecting portion 1370 and/or towards the reference electrode 1368. The same holds for the reference electrode portions 1371 which extend towards the sensing electrode 1366 and/or away from the connecting portion 1372. Electrode portions 1371 are formed by the electrode track 1362. The arrangement of the electrode portions 1366 and 1371 can establish the shown meandering shape of the area of the flexible conductor carrier 1350 separating the reference electrode 1368 and the sensing electrode 1366 in the setting sensing region 1310. The sensing electrode and the reference electrode may form the shown comb-like structure in sensing region 1310.

In the sensing region 1310 the electrode system 1345 comprises a plurality of cutouts or slits 1384. The cutouts or slits 1384 extend through the entire electrode system such that they are accessible from two opposite sides of the electrode system 1345. The cutouts or slits 1384 are expediently provided to attach or connect the electrode system and, particularly, the sensing region 1310 to the user interface member part or chassis 1670 of the associated electronic system 1000 (see FIG. 15C, for example). The cutouts or slits 1384 may be equidistantly disposed along the extension of the sensing region 1310, e.g. from the first end 1356 to the second end 1358. The cutouts or slits 1384 may be delimited by the flexible conductor carrier and/or by one electrode track or by more electrode tracks along their entire circumference. The cutouts or slits may have varying lengths or equal lengths (in the depicted embodiment, the length varies; preferably any two arbitrary adjacent cutouts have different lengths, where the length may be measured perpendicularly relative to the main extension direction of the sensing region 1310). The cutouts or slits 1384 may be oriented such that, once the electrode system is applied in the electronic system 1000, the main extension direction of the cutouts or slits is oriented along an axis running from the proximal end to the distal end. One or more of the cutouts or slits 1384 may be arranged such that they are restricted to a subregion of the sensing region 1310 where no electrode portion is arranged and/or to a subregion where one of the electrode portions 1369 and 1371 is arranged. The cutouts or slits may be fully or partially surrounded and/or delimited by one of the electrode portions 1369 and 1371. In the depicted embodiment, the cutouts or slits through the sensing electrode portions 1369 are only partially surrounded by the electrode portion 1369 (e.g. between two adjacent electrode portions 1369) whereas the cutouts or slits through the reference electrode portions 1371 are fully surrounded by an associated electrode portion 1371. This could also be reversed or both electrode portions can either partially or fully surround a respective one of the cutouts or slits 1384.

The electrode system 1345 further comprises a connector region 1386. The connector region 1386 is connected to the sensing region 1310 via the, expediently flexible, connection region 1380. The connector region 1386 is movable relative to the sensing region 1310. The electrode track 1362 and/or the electrode track 1364 extends from the sensing region 1310 via the connection region 1380 to the connector region 1386. The connection region 1380 has a width which is smaller than the width of the sensing region 1310 and/or than the width of the connector region 1386. The outer circumference of the connector region 1386 or sections thereof may have a fully or partially circular shape or may define a circular enveloping curve.

The connector region 1386 is arranged between the sensing region 1310 and the contact connection region 1352 (as seen along the flexible conductor carrier 1350). The contact connection region 1352 is connected to the connector region 1386 via a further connection region 1382 of the electrode system 1345. The further connection region 1382 may extend away from the connector region 1386 at a location diametrically opposite from the connection region 1380. The connector region 1386 or the connection region 1380 connecting the connector region 1386 to the sensing region 1310 is positioned between the ends 1356 and 1358, e.g. centrally or in the middle and/or remote from both ends. In the connector region 1386, a further electrode track 1378 is arranged. The electrode track 1378 is accessible in the contact connection region 1352. The electrode tracks 1362 and 1364 are as well accessible in the contact connection region 1352 to facilitate contact connection to the electrode arrangement in just one contact connection region. The electrode track 1378, however, does not extend into the sensing region 1310. Rather, track 1378 is restricted to the connector region 1386, the contact connection region 1352 and the connection region 1382.

The electrode track 1378 provides a connector electrode 1388. The connector electrode 1388 is provided as an interface of the electrode system to a separate sensing electrode. In the depicted embodiment the electrode is provided by the separate electrode or spring 1390 (see FIG. 15C), e.g. a power supply spring. The connector electrode 1388, when the electrode system 1345 is arranged in the electronic system, is provided to face towards a distally directed surface of a wall of the user interface member body 1605, wherein the wall delimits the user interface member body proximally. The size of the connector region 1386 and/or the size of the connector electrode 1388 may be adjusted to the size of the power supply 1500, e.g. a coin cell.

Reference electrode portions 1371 of the electrode track 1362 extend along connector electrode 1388, e.g. along an outer circumference thereof. The reference electrode portions 1371 may have a greater width than the adjacent portions of the electrode track 1362 and/or extend on different, e.g. opposite, sides of the connector electrode 1388. The portions of the electrode tracks in the connector region 1386 aside from the connector electrode 1388 may be covered with insulating material, e.g. with the material of the flexible conductor carrier 1350. Thus, in the connector region 1386, e.g. on that side facing the user interface member body 1605 in the electronic system 1000, only the connector electrode 1388 may be exposed. Having only the connector electrode exposed avoids the risk of a short circuit between different electrode tracks, e.g. caused by the separate sensing electrode 1390. This is why, in FIG. 15A, the connector electrode 1388 is shown hatched which should emphasize that (only) this electrode is accessible or exposed. The connector electrode 1388 has a ring-like shape in the depicted embodiment (while other shapes are generally also possible). The ring has two ends, which may face one another. The ring may extend in the circumferential direction for more than 300°, e.g. more than 350°. The ends of the ring may delimit a passage for a portion of another electrode track (e.g. the electrode track 1362, which defines the reference electrode 1368) from an interior delimited by the connector electrode to the exterior. Having a connector electrode which defines an interior and delimits a passage for another electrode track from the interior to the exterior is not restricted to the ring-like shape.

On that side of the connector region 1380 which is remote from the connector electrode 1388, a terminal portion 1392 of the electrode arrangement, e.g. of the electrode track 1362, may be exposed. Preferably, only the terminal portion 1392 is exposed, e.g. to reduce the risk of short circuits. In the electronic system 1000, the terminal portion 1392 can contact the power supply 1500, e.g. directly (see FIG. 15C). The connector electrode 1388 may be covered with electrically insulating material on that side of the connector region 1386 where the terminal portion 1392 is exposed (accordingly, the terminal portion 1392 and not the connector electrode 1388 is hatched in FIG. 15B which shows the top view onto that side of the connector region 1386 which is remote from the connector electrode 1388 and/or from the proximal end of the user interface member body 1605 in the electronic system). The terminals of the electrode tracks in the contact connection region 1352 are accessible or exposed on the same side of the flexible conductor carrier 1350 as the terminal portion 1392 (which is why the electrode tracks 1362, 1364, and 1378 are hatched in the contact connection region 1352 as well).

FIG. 15C shows a schematic sectional view of the assembled electronic system 1000, which, aside from the adjustments to the electrode system discussed in conjunction with FIG. 15A and FIG. 15B, largely correspond to the system discussed in conjunction with FIGS. 10A to 10D. The setting sensing region 1310 is wrapped around or extends around the exterior of the chassis or user interface member part 1670. Protrusions or ridges 1678 of the chassis protrude through the slits 1684 to secure or attach the sensing region 1310 to the chassis 1670. The protrusions or ridges and the cutouts or slits may be configured such that the chassis 1670 can be hooked in the sensing region 1310, e.g. by guiding the protrusions or ridges into the slits or cutouts. In other words, a form-fit connection (e.g. provided by an, preferably axially extending, undercut in the respective protrusion or ridge 1678) may prevent a radial movement of the sensing region 1310 away from the chassis 1670 when the sensing region 1310 is attached to the chassis 1670 and/or an axial movement of the sensing region relative to the chassis. The contact connection region 1352 is electrically conductively connected to a connector 3002 on the conductor carrier 3000, e.g. on side of the conductor carrier 3000 remote from power supply 1500 or a distal side of the conductor carrier 3000. The electrical contact connection between the contact connection region 1352 and the connector 3002 may be formed by a plug-in connector and/or by soldering, for example.

The chassis 1670 with the connected electrode system 1345 can be guided into the user interface member body 1605 and/or is expediently rotationally and/or axially fixed to the body 1605 (e.g. by a snap-fit or welding). The separate sensing electrode or biasing member 1390 biases the connector region 1386 and/or the terminal portion 1392 onto one contact of the power supply, e.g. the ground contact. The associated force may act in the distal direction and/or away from a proximal end surface of the user interface member body. A sensing surface of the electrode 1390 may conform to and/or be in contact with an inner wall of the user interface member body 1605 at the proximal end of the body. The biasing member or separate sensing electrode 1390 biases a contact on the power supply 1500, e.g. the negative contact thereof, onto an electrical terminal 3004 on the conductor carrier 3000. In this way, the contact of the power supply may be connected to the conductor carrier and to the electrode system 1345. Between the conductor carrier 3000 and the power supply 1500 a spacer component 1510 may be arranged (or the spacer can be dispensed with). The motion sensing unit 1200, the electronic control unit 1100 and/or the sensor controller 1340 may be provided in this embodiment as well of course, as may the remaining features of the embodiment of the electronic system described previously.

FIG. 15D shows a subassembly for the electronic system 1000 before insertion into the user interface member body 1605. The subassembly comprises the electronic system 1345, the chassis 1670 and the power supply 1500 and, optionally, the spacer 1510 and/or the carrier 3000 (not explicitly shown). As the electrode system 1345 is connected to the chassis and also to the carrier 3000, the power supply 1500 may be retained reliably in the subassembly, e.g. by the connection portions 1380 and 1382 which may extend axially across a lateral outer surface of the power supply 1500. The connector portion 1386 covers the top surface of the power supply and the terminal portion connects the (other) contact of the power supply to the electrode system.

FIGS. 15E and 15F illustrate an embodiment of the electrode 1390 (biasing member) which can be used in the electronic system. The electrode 1390 has an electrode connection portion 1394. The electrode connection portion 1394 is provided to connect the electrode to the sensor controller 1340 and/or the electrode system 1345, e.g. by bearing on the connector electrode 1388. In the depicted embodiment, the electrode connection portion 1394 is formed by a plurality of (e.g. three) arms or legs. Three legs are particularly advantageous from a stability perspective. The arms are evenly distributed in the circumferential direction. The arms are inclined. The arms extend in the axial, e.g. distal, direction and/or in the radial direction, e.g. radially inwardly. The arms are configured to bear on the connector electrode 1388. The electrode 1390 comprises an electrode sensing portion 1396. The electrode sensing portion 1396 is arranged to contact and/or conform to the inner surface of the wall delimiting the interior of the user interface member body 1605 proximally. The electrode sensing portion 1396 is arranged to enable, facilitate or improve detection of user proximity to the delivery surface 1620 of the user interface member 1600. The sensing portion 1396 has a structured surface (e.g. of a meandering shape in the circumferential direction) so as to maximize the surface area available for (capacitive) sensing. A plurality of circumferentially disposed recesses (e.g. radially oriented recesses) may separate a plurality of protruding electrode portions (e.g. radially protruding electrode portions) which are circumferentially disposed as well. The angular extension of the recesses may be less than the one for the electrode portions. The respective electrode connection portion 1394 may originate at and/or extend from an end (e.g. a radial outer end) of the sensing portion 1396, preferably at and/or from a protruding portion thereof.

The electrode or biasing member 1390 is expediently elastically deformed in the situation shown in FIG. 15C, e.g. to provide a distally directed restoring force. Hence, it may bias components which are disposed distally from the biasing member or electrode 1390 into mechanical contact with one another or strengthen the mechanical and/or electrical contact. For example, one or more or all of the components of the following pairs of components may be biased into mechanical contact with one another by the biasing member or electrode 1390:
terminal portion 1392 and power supply 1500 (e.g. at the proximal side of the power supply, such as the ground or positive contact);
power supply 1500 (e.g. at the distal side of the power supply, such as the negative contact) and electrical terminal 3004;
conductor carrier 3000 and a seat or bearing surface for the carrier in the chassis 1670 (not explicitly shown);
motion sensing unit 1200, e.g. a light transition surface thereof, and the light guide 1676, e.g. a light transition surface thereof (which may comprises a light beam shaping portion formed like a lens). This may improve the optical coupling between the light guide 1676 and the motion sensing unit 1200, which may operate optically.

The electrode system 1345, e.g. in the end region at one end (not depicted in FIGS. 15A and 15B) or both ends 1356 and 1358 (as depicted)) has one or more cutouts 1398. When the sensing region 1310 has been connected to the chassis, the cutout(s) may define or delimit a passage for the contact connection region 1352 into the interior of the chassis, e.g. for connection to the connector 3002 (see FIG. 15D).

Having the electrode system extending over the power supply in the subassembly depicted in FIG. 15D has the advantage that a dedicated clip for securing the power supply in a subassembly can be avoided. Rather, the power supply 1500 is secured to the chassis/user interface member part 1670 via the electrode system 1345.

We note that the layout of the electrode system or the electronic system proposed in FIGS. 15A to 15F and/or the arrangement of the electrode system in the electronic system could also be applied for a system which does not use a separate sensing electrode. In this case, the biasing member is expediently position distally from the region 1386 which then serves as delivery sensing region instead as connector region. Likewise, the layout of the electronic system in FIGS. 10A to 10D could also be adjusted to employ a separate sensing electrode for monitoring proximity to the delivery surface 1620 of the user interface member 1600.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(w-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

### REFERENCE NUMERALS

1 injection device, drug delivery device or device unit
10 housing
12 dosage knob
11 injection button
13 window
14 container
15 needle
16 inner needle cap
17 outer needle cap
18 cap
70 sleeve
71a-c formation
1000 electronic system
1100 electronic control unit
1200 motion sensing unit
1310 setting sensing region
1320 delivery sensing region
1330 user proximity detection unit
1340 sensor controller
1345 electrode system
1350 flexible conductor carrier
1352 contact connection region
1354 portion
1356 end
1358 end
1359 mechanical portion
1360 electrode arrangement
1361 angular clearance
1362 electrode track
1364 electrode track
1366 sensing electrode
1368 reference electrode
1369 sensing electrode portion
1370 connecting portion
1371 reference electrode portion
1372 connecting portion
1373 cutout
1374 terminal
1375 terminal
1376 sensing electrode
1378 electrode track
1379 terminal
1380 connection region
1381 biasing member
1382 connection region
1383 end
1384 slit
1386 connector region
1388 connector electrode
1390 electrode
1392 terminal portion
1394 electrode connection portion
1396 electrode sensing portion
1398 cutout
1400 communication unit
1500 power supply
1510 spacer component
1515 orientation feature
1520 power supply electrode
1530 contact portion
1540 biasing portion
1550 ring portion
1600 user interface member
1605 user interface member body
1610 setting surface
1615 connection feature
1617 surface structure
1616 structure element
1618 transition region
1620 delivery surface
1630 part
1670 chassis
1672 rigid portion
1674 deformable portion
1675 receiving space
1676 light guide
1678 ridge
1680 flange
1681 snap feature
1682 recess
1683 slot
1684 corresponding feature
1700 connection detection unit
1710 switch
3000 conductor carrier
3002 connector
3004 terminal
4000 object
A axis
C line
L length
R area
P plane

1.-21. (canceled)

22. A sensor electrode system for an electronic system, the sensor electrode system comprising:
a flexible conductor carrier which is electrically insulating; and
an electrode arrangement, wherein the electrode arrangement comprises at least two electrically conductive electrode tracks, wherein the at least two electrically conductive electrode tracks extend along the flexible conductor carrier, wherein the at least two electrically conductive electrode tracks are electrically separated from each other along the flexible conductor carrier, wherein one of the at least two electrically conductive electrode tracks forms a sensing electrode, and wherein the sensing electrode extends in a sensing region of the flexible conductor carrier.

23. The sensor electrode system of claim 22,
wherein the sensor electrode system is elastically deformable.

24. The sensor electrode system of claim 22,
wherein one of the at least two electrically conductive electrode tracks of the electrode arrangement forms a reference electrode, wherein the reference electrode extends along the sensing electrode in the sensing region of the flexible conductor carrier, wherein the reference electrode and the sensing electrode are configured to be provided with different electrical potentials when the sensor electrode system is operated.

25. The sensor electrode system of claim 24,
wherein, in the sensing region, the reference electrode has a plurality of reference electrode portions and the sensing electrode has a plurality of sensing electrode portions, wherein the sensing electrode portions and the reference electrode portions are alternatingly disposed.

26. The sensor electrode system of claim 25,
wherein an end of at least one sensing electrode portion faces towards the reference electrode, and wherein an end of at least one reference electrode portion faces towards the sensing electrode.

27. The sensor electrode system of claim 22,
wherein the sensing region is configured to conform to and/or extend along a circumferentially disposed sensing surface of a user interface member of an electronic system, and wherein the sensor electrode system is configured such that one reference electrode portion and one sensing electrode portion are disposed at opposite locations when the sensor electrode system is provided in the electronic system.

28. The sensor electrode system of claim 22
wherein the sensing electrode is a first sensing electrode and the sensing region is a first sensing region, wherein one of the at least two electrically conductive electrode tracks of the electrode arrangement forms a second sensing electrode which extends in a second sensing region of the flexible conductor carrier, and wherein the flexible conductor carrier is configured such that the first sensing region and the second sensing region are movable relative to one another.

29. The sensor electrode system of claim 24,
wherein the sensing electrode is a first sensing electrode and the sensing region is a first sensing region, wherein one of the at least two electrically conductive electrode tracks of the electrode arrangement forms a second sensing electrode which extends in a second sensing region of the flexible conductor carrier, and wherein the flexible conductor carrier is configured such that the first sensing region and the second sensing region are movable relative to one another,
wherein the reference electrode extends along the first sensing electrode in the first sensing region and along the second sensing electrode in the second sensing region.

30. The sensor electrode system of claim 22,
a) wherein the sensing electrode is a first sensing electrode and the sensing region is a first sensing region, wherein one of the at least two electrically conductive electrode tracks of the electrode arrangement forms a second sensing electrode which extends in a second sensing region of the flexible conductor carrier, and wherein the flexible conductor carrier is configured such that the first sensing region and the second sensing region are movable relative to one another, wherein the flexible conductor carrier comprises a contact connection region, and wherein at least one of the reference electrode, the sensing electrode, or the second sensing electrode is configured to be contacted electrically in the contact connection region, and/or
b) wherein one of the at least two electrically conductive electrode tracks forms a connector electrode, which extends in a connector region of the flexible conductor carrier, wherein the connector region is configured to electrically conductively connect a separate sensing electrode to the electrode arrangement.

31. An electronic system for a drug delivery device, the electronic system comprising:
a user interface member which includes at least one exterior operation surface which is arranged to be manipulated by a user for a dose operation;
a user proximity detection unit which is arranged and configured to detect whether the user is close to or touches the exterior operation surface, wherein the user proximity detection unit comprises a sensor electrode system of claim 22; and
an electronic sensor controller, the electronic sensor controller being electrically conductively connected to the sensor electrode system.

32. The electronic system of claim 31,
a) wherein the sensing region is biased towards the exterior operation surface, and wherein a biasing member is provided in the electronic system for biasing the sensing region towards the exterior operation surface, and/or
b) wherein the electronic sensor controller is arranged on a carrier, wherein the carrier is secured to a user interface member part of the user interface member, wherein the sensing region extends along an outer surface of the user interface member part, and wherein a contact connection region extends inwardly and is conductively connected to the electronic sensor controller.

33. The electronic system of claim 32,
wherein the electronic system comprises a power supply, wherein the power supply is arranged between the carrier and a portion of the flexible conductor carrier, wherein the flexible conductor carrier is secured to the user interface member part, wherein a connection region of the flexible conductor carrier extends from the portion of the flexible conductor from a side of the power supply which is remote from the carrier towards the carrier, and wherein the connection region is mechanically and/or electrically connected to the carrier.

34. The electronic system of claim 31,
a) wherein the sensing electrode is assigned to a setting surface of the user interface member, and/or wherein the sensing electrode is assigned to a delivery surface of the user interface member, and/or
b) wherein the sensing electrode is a first sensing electrode and the sensing region is a first sensing region, wherein one of the at least two electrically conductive electrode tracks of the electrode arrangement forms a second sensing electrode which extends in a second sensing region of the flexible conductor carrier, and wherein the flexible conductor carrier is configured such that the first sensing region and the second sensing region are movable relative to one another, wherein the first sensing electrode is assigned to a setting surface of the user interface member, and wherein the second sensing electrode is assigned to a delivery surface of the user interface member.

35. The electronic system of claim 31,
wherein the sensing electrode is a first sensing electrode and the sensing region is a first sensing region, wherein one of the at least two electrically conductive electrode tracks of the electrode arrangement forms a second sensing electrode which extends in a second sensing region of the flexible conductor carrier, and wherein the flexible conductor carrier is configured such that the first sensing region and the second sensing region are movable relative to one another, and wherein the first sensing region and the second sensing region are configured to be assigned to exterior operation surfaces which face in different directions.

36. The electronic system of claim 31,
wherein the exterior operation surface is a setting surface for a dose setting operation and faces in a radial direction.

37. The electronic system of claim 31,
wherein the exterior operation surface is configured such that the user proximity detection unit has a higher sensitivity for softer or more flexible conductive objects that conform to the exterior operation surface relative to harder or stiffer conductive objects that do not conform as easily to the exterior operation surface.

38. A drug delivery device comprising a sensor electrode system, the sensor electrode system comprising:
a flexible conductor carrier which is electrically insulating; and
an electrode arrangement, wherein the electrode arrangement comprises at least two electrically conductive electrode tracks, wherein the at least two electrically conductive electrode tracks extend along the flexible conductor carrier, wherein the at least two electrically conductive electrode tracks are electrically separated from each other along the conductor carrier, wherein one of the at least two electrically conductive electrode tracks forms a sensing electrode, and wherein the sensing electrode extends in a sensing region of the flexible conductor carrier.

39. The drug delivery device of claim 38, further comprising a reservoir with a drug.

40. A method comprising dispensing a drug using a drug delivery device according to claim 39.

41. A method of manufacturing an electronic system for a drug delivery device, the method comprising:
providing an electrode system, the electrode system comprising a sensor electrode system, wherein the sensor electrode system comprises:
a flexible conductor carrier which is electrically insulating; and
an electrode arrangement, wherein the electrode arrangement comprises at least two electrically conductive electrode tracks, wherein the at least two electrically conductive electrode tracks extend along the flexible conductor carrier, wherein the at least two electrically conductive electrode tracks are electrically separated from each other along the conductor carrier, wherein one of the at least two electrically conductive electrode tracks forms a sensing electrode, and wherein the sensing electrode extends in a sensing region of the flexible conductor carrier, and
wherein the electrode system comprises a flexible conductor carrier which is electrically insulating and an electrode arrangement, wherein the electrode arrangement comprises at least one electrically conductive electrode track extending along the flexible conductor carrier;
providing a user interface member part;
deforming the electrode system such that a surface of the flexible conductor carrier extends along the user interface member part; and
connecting the at least one electrically conductive electrode track conductively with an electronic unit to form the electronic system.
